(19) United States
(12) Patent Application Publication
(10) Pub. No.: US 2025/0360005 A1
(43) Pub. Date: Nov. 27, 2025

(54) Adjustment and Metrology Apparatuses and Methods for Medical Devices

(71) Applicant: LIMBER Prosthetics & Orthotics Inc., San Diego, CA (US)

(72) Inventors: Joshua PELZ, San Diego, CA (US); Herbert J. BARRACK, San Diego, CA (US); Luca DE VIVO, Oahu, HI (US); Edward DERMARDIROSIAN, San Diego, CA (US); Bradley Carroll RICE, Carlsbad, CA (US); Giacomo Michael Pasquale STROLLO, San Diego, CA (US)

(73) Assignee: LIMBER Prosthetics & Orthotics Inc., San Diego, CA (US)

(21) Appl. No.: 19/210,350

(22) Filed: May 16, 2025

Related U.S. Application Data
(60) Provisional application No. 63/650,040, filed on May 21, 2024.

(51) Int. Cl. A61F 2/50; A61F 2/60

(52) U.S. Cl. CPC A61F 2/5046; A61F 2/60; A61F 2002/5052

(57) ABSTRACT

Adjustment and metrology systems include a first securing component configured to hold a first region of the medical device. A second securing component is configured to hold a second region of the medical device, the first securing component and the second securing component being spaced apart in a Z-direction. A plurality of positioning mechanisms is coupled to at least one of the first securing component or the second securing component, the plurality of positioning mechanisms being configured to provide a combined total of at least six degrees of freedom between the first securing component and the second securing component, wherein the degrees of freedom comprise translational movement in an X-direction, a Y-direction, and the Z-direction and rotational movement about an X-axis, a Y-axis, and a Z-axis. A heater is positioned between the first securing component and the second securing component.

### RELATED APPLICATIONS

This application claims priority to U.S. Provisional Patent Application No. 63/650,040, filed on May 21, 2024, and entitled “Thermoforming Apparatus”; the contents of which are hereby incorporated by reference.

### BACKGROUND

Prostheses and orthoses are medical devices that are used to improve function in patients who have a body part that is not operating properly or is missing. As an example, prosthetic limbs may serve as artificial replacements for legs or arms of amputees. These medical devices must be custom fit, requiring careful design, alignment and adjustment for each individual. Conventional vertical transfer jigs involve a vertical post with typically three locking arms that slide along the post. The arms are used to hold prosthetic components (e.g., socket, knee, pylon, ankle, foot for a prosthetic leg) while the components are assembled and aligned for a patient.

Another type of adjustment that can be made for prosthetic and orthotic devices is thermoforming. Thermoforming is a technique where a plastic material is heated to a pliable temperature and formed into a shape, for example by conforming a heated sheet of a thermoplastic material onto a mold. Thermoplastic materials become pliable at elevated temperatures and then solidify upon cooling. Thermoforming can be used to adjust the fit and alignment of many types of prosthetic and orthotic devices made of thermoplastic materials. In such cases, thermoforming can involve the heating and shaping of the thermoplastic material to provide final adjustments in accordance with a patient's needs. Conventional thermoforming techniques for prosthetic and orthotic devices involve making adjustments manually or by using tools to reshape portions of the prosthesis.

### SUMMARY

In some aspects, a metrology and adjustment system includes a first securing component configured to hold a first region of the medical device. A second securing component is configured to hold a second region of the medical device, the first securing component and the second securing component being spaced apart in a Z-direction. A plurality of positioning mechanisms is coupled to at least one of the first securing component or the second securing component, the plurality of positioning mechanisms being configured to provide a combined total of at least six degrees of freedom between the first securing component and the second securing component, wherein the six degrees of freedom comprise translational movement in an X-direction, a Y-direction, and the Z-direction and rotational movement about an X-axis, a Y-axis, and a Z-axis. A heater is positioned between the first securing component and the second securing component.

In some aspects, a metrology and adjustment apparatus for a medical device includes a first securing component configured to hold a first region of the medical device, and a second securing component configured to hold a second region of the medical device. A linear translation stage is coupled to the second securing component, the linear translation stage configured to move the second securing component in an X-direction and in a Y-direction that is perpendicular to the X-direction. A Z-rotation mechanism is configured to rotate the second securing component about a Z-axis that is perpendicular to the X-direction and the Y-direction. A Z-translation stage is coupled to the first securing component, the Z-translation stage configured to move the first securing component in a Z-direction that is perpendicular to the X-direction and the Y-direction. An X-rotation mechanism is configured to rotate the first securing component about an X-center of rotation having an X-axis that is oriented in the X-direction. A Y-rotation mechanism is configured to rotate the first securing component about a Y-center of rotation having a Y-axis that is oriented in the Y-direction. An arm couples the first securing component to at least one of the X-rotation mechanism or the Y-rotation mechanism, wherein the arm is configured to allow adjustment of a distance between the first securing component and at least one of the X-center of rotation and the Y-center of rotation.

In some aspects, a metrology and adjustment apparatus for a medical device, the apparatus includes a first securing component configured to hold a first region of the medical device. A second securing component is configured to hold a second region of the medical device, the first securing component and the second securing component being spaced apart in a Z-direction. A plurality of positioning mechanisms is configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other; wherein a distance between the first securing component and a center of rotation of the first securing component is adjustable. In some cases, a heater is positioned between the first securing component and the second securing component.

In some aspects, a method of adjusting a medical device includes: providing a metrology and adjustment apparatus having a first securing component, a second securing component, and a heater between the first securing component and the second securing component, wherein the first securing component and the second securing component are spaced apart in a Z-direction; securing a first region of the medical device with the first securing component; securing a second region of the medical device with the second securing component; positioning a heating zone of the medical device within the heater; heating the heating zone of the medical device; and adjusting a plurality of positioning mechanisms coupled to the first securing component and the second securing component to thermoform the medical device. The plurality of positioning mechanisms are configured to provide: i) translation in an X-direction, a Y-direction, and the Z-direction for a relative position of the first securing component and the second securing component with respect to each other; and ii) at least two degrees of freedom of angular orientation between the first securing component and the second securing component relative to each other.

In some aspects, a method of adjusting a medical device includes: a) providing a metrology and adjustment apparatus having: a first securing component; a second securing component, wherein the first securing component and the second securing component are spaced apart in a Z-direction; a plurality of positioning mechanisms coupled to the first securing component and the second securing component, wherein the plurality of positioning mechanisms are configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other; and an arm coupled to the first securing component, wherein the arm is configured to allow adjustment of a distance between the first securing component and a center of rotation of the first securing component; b) securing a first region of the medical device with the first securing component; c) securing a second region of the medical device with the second securing component; and d) adjusting the medical device using the plurality of positioning mechanisms.

### BRIEF DESCRIPTION OF THE DRAWINGS

FIG. 1 is a side view of a transtibial prosthetic device, as an example of a medical device.

FIGS. 2A-2B are front and rear views, respectively, of a medical device mounted in a thermoforming apparatus, in accordance with some aspects.

FIG. 3A is a perspective view of a first securing component, in accordance with some aspects.

FIG. 3B is a perspective view of a medical device mounted in the first securing component of FIG. 3A, in accordance with some aspects.

FIG. 3C is a perspective view of a transtibial prosthesis with alignment features, in accordance with some aspects.

FIGS. 4A-4B are perspective views of example configurations for first or second securing components, in accordance with some aspects.

FIG. 5A is a perspective view of a second securing component, in accordance with some aspects.

FIG. 5B is a front view of the second securing component of FIG. 5A holding a medical device, in accordance with some aspects.

FIGS. 6A and 6B are perspective views of a thermoforming apparatus, in accordance with some aspects.

FIGS. 6C-6D are views of components involved with adjusting the position of a securing component, in accordance with some aspects.

FIG. 7 is a perspective view of a linear translation stage and a Z-rotation mechanism, in accordance with some aspects.

FIG. 8 is a perspective view of an X-rotation mechanism, in accordance with some aspects.

FIG. 9 shows a front view of a Y-rotation mechanism, in accordance with some aspects.

FIGS. 10A-10C are various views of a Z-translation stage, in accordance with some aspects.

FIG. 11 is a front view of a heater for a thermoforming apparatus, in accordance with some aspects.

FIG. 12 is a side view of a heater with a heat source, in accordance with some aspects.

FIGS. 13A-13B show internal and external views of a heater, in accordance with some aspects.

FIG. 14A is a flowchart representing methods for measuring and adjusting a medical device, in accordance with some aspects.

FIG. 14B is a flowchart representing methods for measuring and adjusting a medical device without thermoforming, in accordance with some aspects.

FIG. 15 is a block diagram of a metrology and adjustment system that includes automated control and reporting capabilities, in accordance with some aspects.

FIG. 16 is a schematic illustrating a centroid of rotation for the metrology and adjustment apparatus, in accordance with some aspects.

FIG. 17 is a simplified schematic diagram showing an example computer system for use in the methods and systems of the present disclosure.

### DETAILED DESCRIPTION

The present disclosure describes an adjustment and metrology apparatus that uniquely facilitates measurements and/or adjustments of a prosthetic or orthotic device in multiple degrees of freedom and in a precise and controllable manner. The apparatus-which may also be referred to as a fixture or system-beneficially enables a practitioner (e.g., a prosthetist) to quantify and document the changes to the medical device (e.g., prosthesis or orthosis). These quantified measurements are important for documenting not only the patient's history, but also to provide justification to insurance regarding the patient's status. Conventional adjustment and metrology techniques are typically qualitative in nature and consequently are difficult to measure and document.

The adjustment and metrology apparatuses and methods described herein may include a heater between a first securing component and a second securing component, such that the apparatus may serve as a thermoforming system to make adjustments to a medical device (e.g., a prosthesis or orthosis). The adjustments may involve altering the position or shape of a component within the medical device to improve the fit, balance, function, or other aspect for the patient. In scenarios where a heater is not included, the apparatuses may be used to adjust and/or measure medical devices, where the medical devices may or may not be thermoformable.

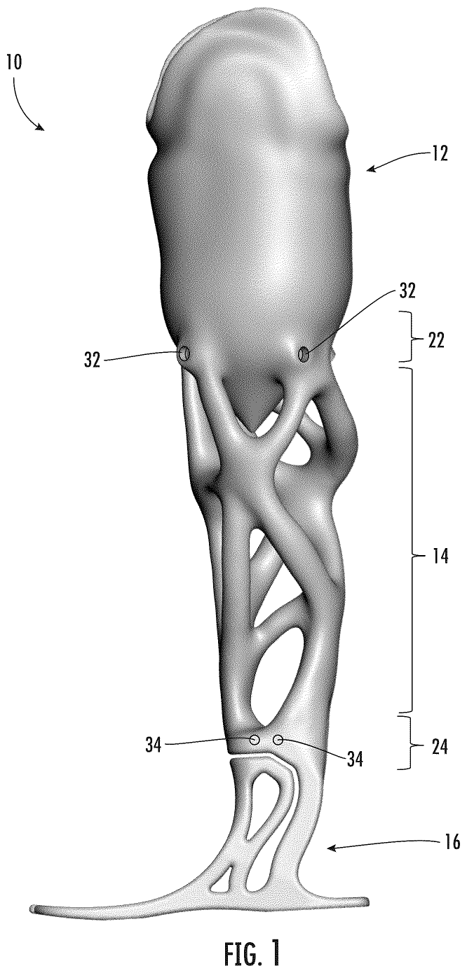

The adjustment and metrology apparatuses and methods may be applied to medical devices such as prostheses and orthoses. In this disclosure, a medical device 10 as shown in FIG. 1 is used for illustration, where medical device 10 is a transtibial prosthesis. In other examples, the prosthetic device may be for an upper portion of a leg (transfemoral) such as hip to knee. In other examples, the prosthesis may be for a portion of an arm (upper extremity) instead of a leg (lower extremity), such as for a forearm (e.g., wrist to elbow or hand to elbow) or for an upper arm (e.g., shoulder to elbow). In further examples, the medical device may be an orthotic device (e.g., knee or ankle brace) for any of these regions including upper extremities or lower extremities.

Medical device 10 includes a socket 12, a pylon 14, and a foot 16. Socket 12 is shaped to attach to a residual limb of the patient. The pylon 14 in this example is made of a thermoplastic that enables the pylon 14 to be thermoformed. Example thermoplastic materials that may be used include polycarbonate (PC), polyethylene (PE), polypropylene (PP), polyethylene terephthalate glycol (PETG), polyamide (PA), acrylonitrile butadiene styrene (ABS), and thermoplastic polyurethane (TPU).” The thermoplastic material permits alignment adjustments (e.g., during a patient fitting) by heating the pylon, such that the heating zone of the medical device 10 in this case is the pylon 14. In this example, medical device 10 is fabricated as a unitary single piece from a thermoplastic material, with the pylon 14 having an endoskeletal (i.e., adjustable) design. In particular, the endoskeletal design of the prosthetic device 10 comprises interconnected supports that form a truss structure, where the interconnected supports may curve diagonally (e.g., three-dimensionally), providing deformation without buckling and consequently enabling alignment adjustment of one or more portions of the prosthesis by thermoforming while maintaining its structural integrity. In some cases, the medical device 10 may be 3D printed as a unibody (i.e., single piece) structure. Details of the prosthetic device shown in FIG. 1 may be found in U.S. Pat. Nos. 11,938,043 and 12,208,025, entitled “Unibody Endoskeletal Transtibial Prosthetic Devices and Digital Fabrication Workflow,” which are hereby incorporated by reference.

A first region 22 is an area (e.g., joint) between the socket 12 and the pylon 14. A second region 24 is an area (e.g., joint) between the pylon 14 and the foot 16. The first region 22 and second region 24 in this disclosure serve as holding areas during thermoforming of the pylon 14. In some aspects, the socket 12 and/or foot 16 may not be included as part of the medical device 10 during thermoforming, in which case the first region 22 and/or second region 24 may be ends of the pylon 14. In some aspects, other sections of the medical device 10 may be targeted for thermoforming (e.g., entire medical device 10, or pylon and foot, or socket and pylon), in which case first region 22 and second region 24 would be the ends of the targeted portion. In other aspects where the medical device 10 is for a different limb than the tibia, pylon 14 may represent, for example, a humerus, a forearm, or femur. In those cases, the first region 22 and second region 24 may be areas near ends of the pylon 14 or at joints between. For example, the first region 22 or second region 24 may be in areas corresponding to a knee, a hip, a shoulder, or a wrist.

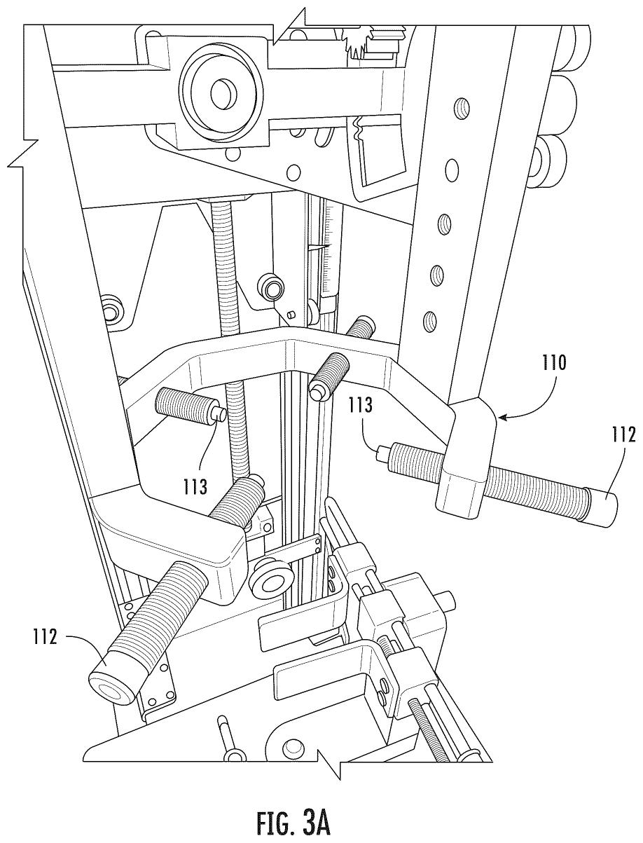

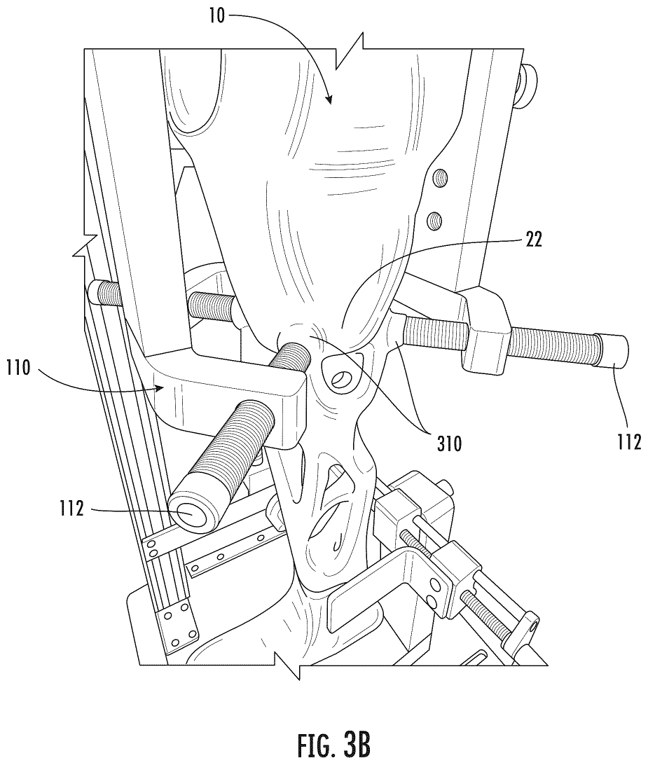

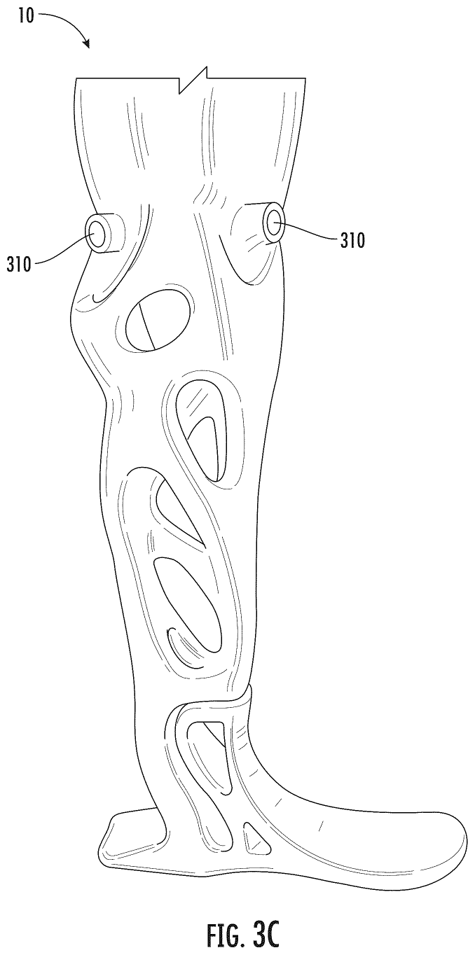

Also shown in FIG. 1 are alignment features 32 in the first region 22 and the alignment features 34 in the second region 24. Alignment features 32 and 34 are features incorporated into the medical device 10 to serve as datum points for measuring and/or adjusting the medical device 10. In this illustration, alignment features 32 and 34 are circular recesses. Further description and examples of alignment features are provided throughout this disclosure, such as in FIGS. 3B-3C.

Although the descriptions in this disclosure shall use a thermoformable prosthetic leg as an example, the systems and methods may be applied to other types of prosthetic and orthotic devices. For example, a transtibial prosthetic limb having a single pipe as the pylon (connecting a socket and a foot) may be adjusted and/or measured using the present systems and methods. The pipe may be made of a material that is not thermoformable (e.g., metal), and adjustments are made by mechanically repositioning components of the prosthetic limb. The adjustments for a non-thermoformable device may include altering the positions/alignments of the socket, pylon, and/or foot relative to each other, such as an angle of the pylon relative to the socket or to a vertical axis.

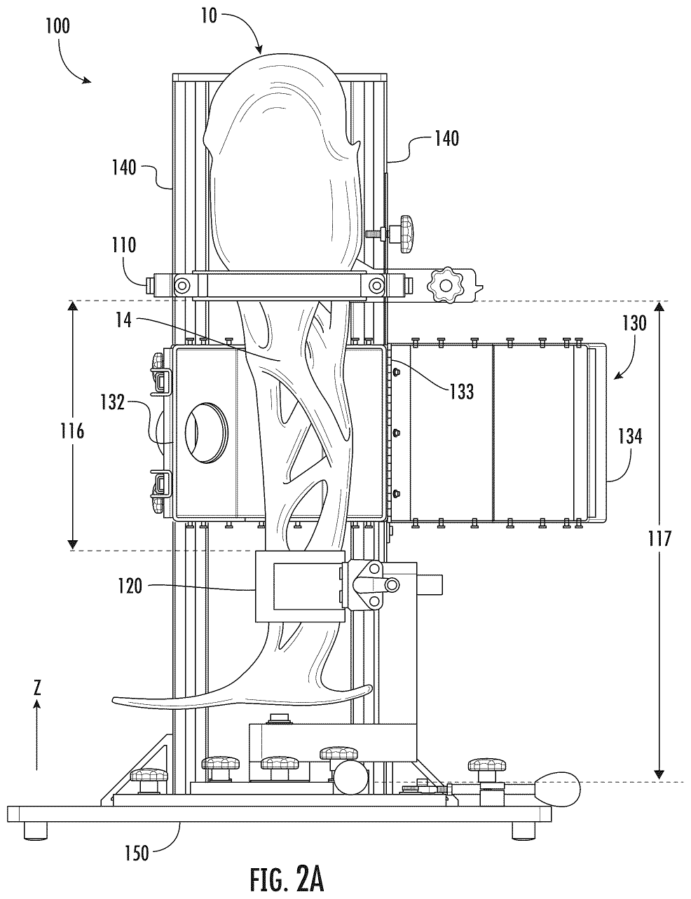

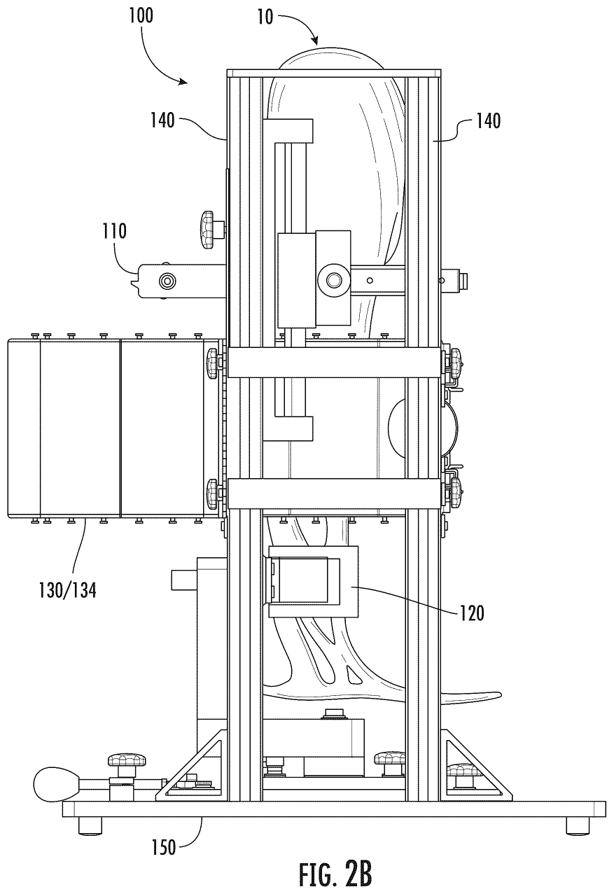

FIGS. 2A and 2B show front and rear views, respectively, of medical device 10 mounted in a thermoforming apparatus 100, in accordance with some aspects. Thermoforming apparatus 100 includes a first securing component 110 configured to hold first region 22 of the medical device 10, and a second securing component 120 configured to hold second region 24 of the medical device 10. Thermoforming apparatus 100 may include a heater 130 that is positioned between the first securing component 110 and the second securing component 120. The heater 130 is shown in an open position in FIG. 2A, where the pylon 14 is in front of a back half 132 of the heater 130. A front half 134 of the heater 130 can be swung onto the back half 132 to enclose the pylon 14, such as via hinges 133 that connect front half 134 to back half 132. The first securing component 110 is mounted on a pair of rails 140 in this example, with the rails 140 being supported by a base 150. Second securing component 120 is coupled to base 150, and heater 130 is coupled to first securing component 110. A distance 116 between the first securing component 110 and the second securing component 120 is adjustable by moving first securing component 110 along the linear rails 140. Example dimensions for the thermoforming apparatus 100 may be, for example, a distance 116 of about 150 mm to about 250 mm, or about 200 mm to about 220 mm between the first securing component 110 and the second securing component 120; and a distance 117 of about 350 mm to about 450 mm, or about 390 mm to about 410 mm) between the base 150 and the first securing component 110.

In some aspects, the heater 130 may be omitted from thermoforming apparatus 100 such that the apparatus may be used as a metrology and adjustment apparatus. For example, the apparatus 100 without the heater 130 (or without using the heater 130 if present) may be used to measure alignment of components within the medical device 10 and/or to make adjustments to relative position or angles of the components.

FIG. 3A is a perspective view of the first securing component 110 which serves as an upper clamp to hold an area near the top end of the medical device (first region 22), in accordance with some aspects. FIG. 3B shows a perspective view of the medical device 10 mounted in first securing component 110. Note that the securing mechanisms for first securing component 110 and second securing component 120 described herein may be used interchangeably. That is, examples described for first securing component 110 at a top end of the medical device may be utilized for second securing component 120 at the bottom end of the medical device, or vice versa. Similarly, the mechanisms for first securing component 110 and second securing component 120 may be the same or different from each other, in some cases.

In this example shown in FIGS. 3A-3B, first securing component 110 is a partial ring, open at the front for inserting the medical device 10. The first securing component 110 is configured with multiple holding elements 112 (e.g., illustrated as threaded bolts) extending through the ring and arranged radially in a plane. The bolts secure the first region 22 (e.g., joint between the socket/pylon) of the medical device 10, such as by advancing or retracting the threaded bolts in or out of the ring that holds the bolts.

In some aspects, the medical device 10 includes alignment features 310 (FIGS. 3B-3C). The ends 113 of the holding elements 112 that contact the medical device 10 can be designed (e.g., shaped and dimensioned) to be seated in the alignment features 310 of medical device 10. In the example of FIGS. 3B-3C, the alignment features 310 are hollow cylindrical features on the surface of the medical device 10, where the interiors and walls of the alignment features 310 provide a location in which the ends 113 of the holding elements 112 may be securely seated. The alignment features 310 can serve as adapters or fixture datums (which may also be referred to as alignment fixture datums) for the holding elements 112. For example, the alignment features 310 may be holes, recesses or concave features shaped to match the shape of the ends of the holding elements 112. Alternatively, the alignment features 310 may be raised features that fit into corresponding features at the ends of the holding elements 112. That is, the male/female arrangement of the ends 113 of the holding elements 112 and of the alignment features 310 may be reversed.

The alignment features 310 may be integrally fabricated as part of the medical device 10, such as being 3D printed as part of a 3D printed medical device, or molded/cast into the material of the medical device. In other aspects, alignment features 310 may be separate components added to the medical device 10 and may be removable. Having the holding elements 112 mate with alignment features 310 of the medical device uniquely enables the medical device to be refitted multiple times over its lifetime as patient deviations arise, such as if the patient changes their gait, or as medical device 10 wears down, or for other reasons.

In the example of FIGS. 3A-3B, the first securing component 110 utilizes four bolts as holding elements 112. The four holding elements 112 are arranged in a plane and spaced approximately 90 degrees apart from each other around the ring. In other examples, other numbers of holding elements 112 may be used, such as one to three, or more than four, and the holding elements may be arranged at different spacings from each other (e.g., 30 degrees to 180 degrees apart around the circumference of the first securing component 110). Furthermore, the bolts may be replaced by other holding elements such as pins. The holding elements 112 may utilize means other than threaded mounts to apply force onto the medical device, such as by being spring-loaded, or pneumatically or hydraulically driven, or by using clips to fix the holding elements in place.

The ends 113 (e.g., tips) of the bolts, pins, or other holding elements 112 may be configured with various shapes to fit into the alignment features 310. For example, the tips/ends may be cylindrical as shown in FIG. 3A to mate with the cylindrical alignment features 310. In other examples, the ends 113 may be concave, convex, flat, spherical, rectangular, conical or other shape to match the shape of the alignment features. In some examples, the ends of the holding elements can have a shape that helps key the first securing component 110 to the alignment features 310. For example, a square end on a pin may be used with a matching shape of the alignment feature 310. The square end (or other shape, e.g., a cross or triangle) helps prevent movement of the medical device with respect to the first securing component 110, such as in rotational or translational movements. The keying shape (and size, in some instances) of the end of the holding element may enable as few as one or two holding elements 112 to be utilized in first securing component 110. For example, in a configuration with one holding element, the holding element 112 may be configured with a hook or may be slid through a channel in the medical device (similar to a locking pin). In various aspects, the holding elements 112 (e.g., bolts, pins) of the first securing component 110 can be custom-formed-such as by molding, casting, or 3D printing—to achieve the desired tip/keying shapes.

In some aspects, the holding elements 112 may serve as datum pins that mate with the alignment features 310 on the medical device 10. The datum pins not only provide a repeatable positioning datum for measurements (e.g., for translation and rotational positions) but also secure the medical device to the thermoforming apparatus 100. The holding elements 112 may be configured to resist rotational moments and linear forces, such as having a shape that mates with an alignment feature on the medical device. The holding elements 112 may also help protect the alignment features 310 by being shaped to mate with the alignment features 310. The holding elements 112 may optionally include a coating or pad where it interfaces with the alignment feature 310 to help prevent damage to the alignment feature.

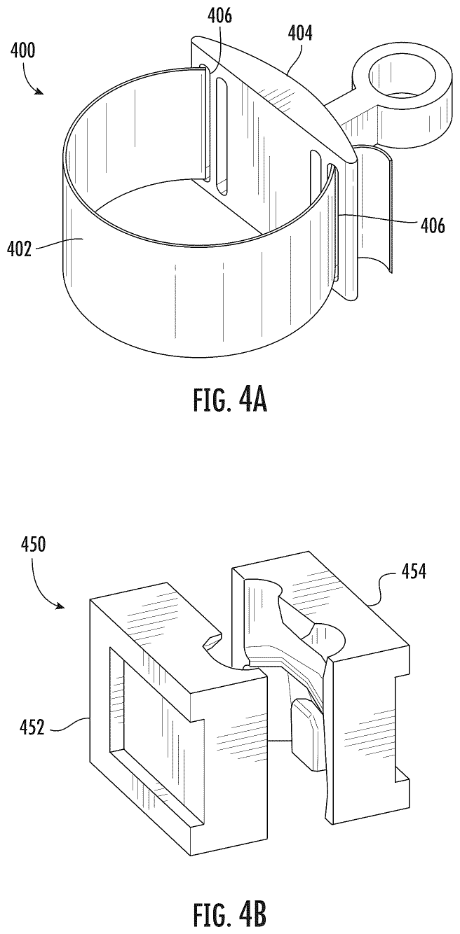

FIGS. 4A and 4B show examples of other configurations for first securing component 110 (or second securing component 120), in which alignment features 310 may not be needed in the medical device. For example, FIG. 4A shows a securing component 400 in which a strap 402 or belt is held by a plate 404 that has slots 406 for holding the ends of the strap 402. The strap 402 (which serves as a holding element) may be wrapped around the first region 22 or second region 24 of the medical device 10 to hold the medical device in place by friction, such as serving as a friction clamp. In one example, the medical device may be secured by tightening the strap 402 around the medical device, where the strap 402 may optionally be made of a material that has a gripping (i.e., high friction) surface. As a result, an alignment feature on the medical device may not be required, although may be used in addition to the strap/belt.

Other example configurations for a securing component include clamps or brackets. The clamps may have conformable surfaces, such as jaws covered with foam, rubber, silicone, or other non-slip material that can conform (i.e., are compliant) to the contour of the medical device. The clamps with conformal surfaces may secure the medical device by friction and/or by mechanically supporting the shape of the medical device. FIG. 4B shows an example of securing component 450 having jaws 452 and 454 for a clamp in which the two jaws have interior surfaces that are shaped to match the contour of a region of the medical device (e.g., first region 22 or second region 24). In particular, the jaws cover a region of the medical device (e.g., first region 22 or second region 24), and the interior surfaces of the jaws have the shape of the region that the clamp will be holding. Because of the shape being customized as the negative of (i.e., by subtraction) of the medical device, the clamps may be made of a hard material and may not need a soft interior surface to contact the medical device. Alternatively, the contoured jaws may include a gripping or conformable material. The shape of the jaws may serve as datum features, due to the surfaces of the jaws (i.e., fixtures) having a geometry that mates with the medical device. In some cases, other numbers of jaws (i.e., fixtures) may be used, such as three, four or more. The jaws may or may not cover the entire perimeter of the medical device.

In some cases, the securing components (e.g., first securing component 110 or second securing component 120) of the present apparatuses and methods may include holding elements that apply clamping forces to secure a medical device in an adjustment and/or metrology apparatus. The holding elements may optionally serve as datum features in addition to providing clamping action. The holding elements may be configured to interface or mate with alignment features on the medical device, thus providing clamping forces by resisting linear forces and rotational moments and/or being a datum feature by providing translational and rotational position registration. For example, the datum features may be longitudinal components (e.g., bolts, pins, rods) having ends shaped and dimensioned to fit with an alignment feature on the medical device as shown in FIGS. 3A-3C. The securing component may have a plurality of holding elements (e.g., datum features) which are discrete components providing discrete datum features and clamping points, such as two, three, or four or more holding elements. In one example, the holding features may be located radially around the securing component, to hold a medical device located in an interior space of the securing component.

In some cases, the securing component may have a holding element such as the strap of FIG. 4A or the jaws of FIG. 4B that applies clamping forces to the medical device over a distributed surface. The holding element with the distributed force may have a conformal, compliant, and/or gripping surface. As an example, the surface may be made of a material that helps hold the medical device by friction. As another example, the surface may help hold the medical device by having a geometry that mates with (e.g., is a negative or inverse geometry of) the region of the medical device that the holding element interfaces with.

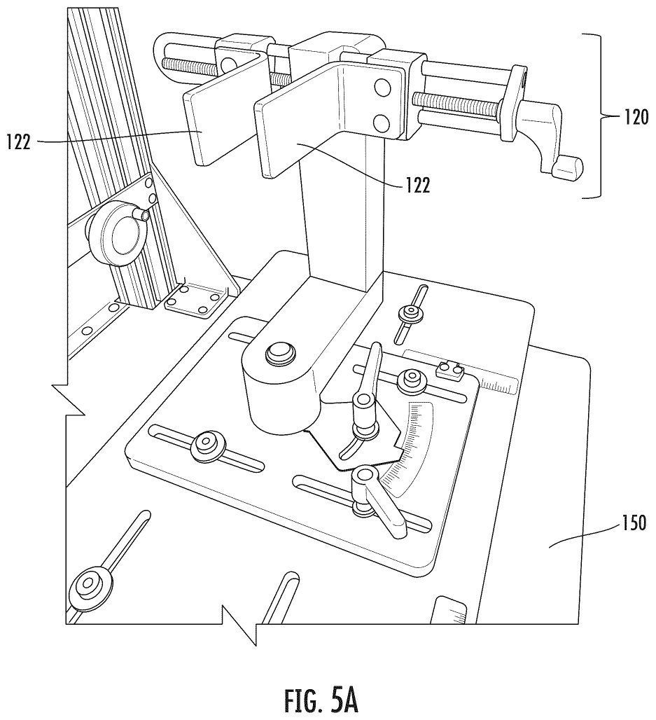

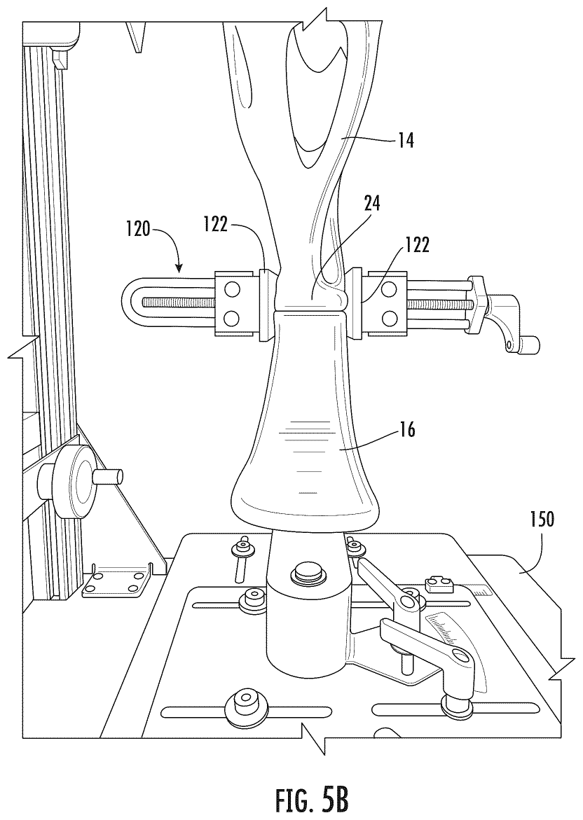

FIG. 5A shows a perspective view of second securing component 120 which serves as a lower clamp of the thermoforming apparatus 100, near the bottom end of the medical device 10 and on base 150. FIG. 5B is a front view of second securing component 120 holding the medical device 10, at second region 24 adjacent to pylon 14. The foot 16 is included in this example, and the second securing component 120 fits above it (e.g., second region 24 being a joint area between pylon 14 and foot 16). The second securing component 120 in this example is configured as a self-centering vise 122 comprising opposing plates (e.g., two plates, jaws, arms, clamps) that are moved together along rails by a rotating crank handle. The plates may include aspects to help secure the medical device, such as by having a textured surface, or a gripping and/or conformable material on the surfaces that contact the medical device. As described above, second securing component 120 may instead be configured with any of the examples described for first securing component 110 such as bolts or pins (and corresponding alignment features in the second region 24 of medical device 10), a strap, or jaws contoured to the shape of the second region 24.

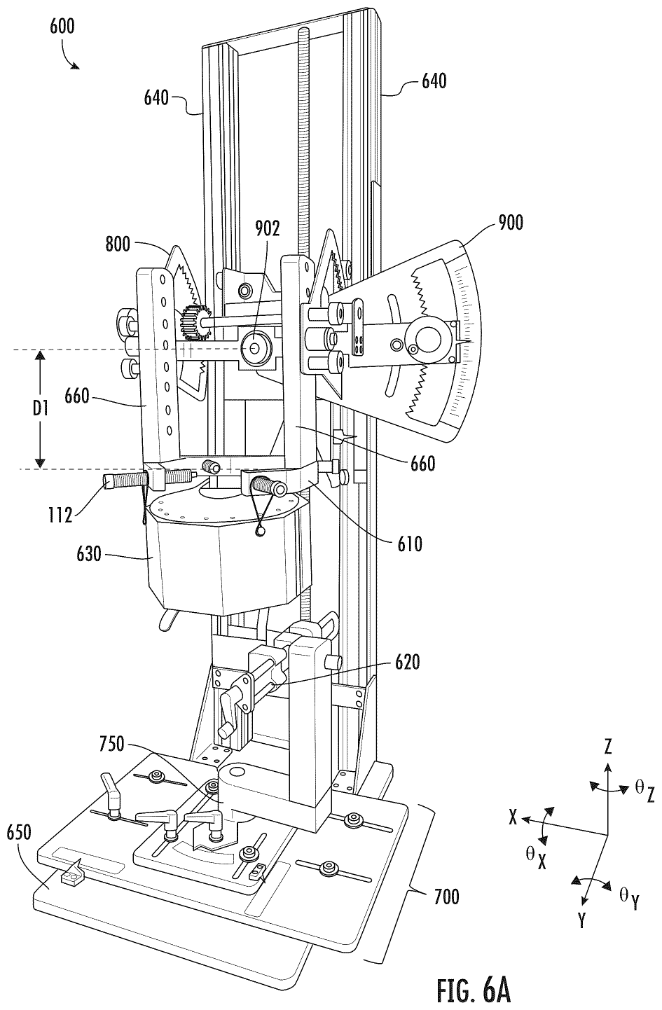

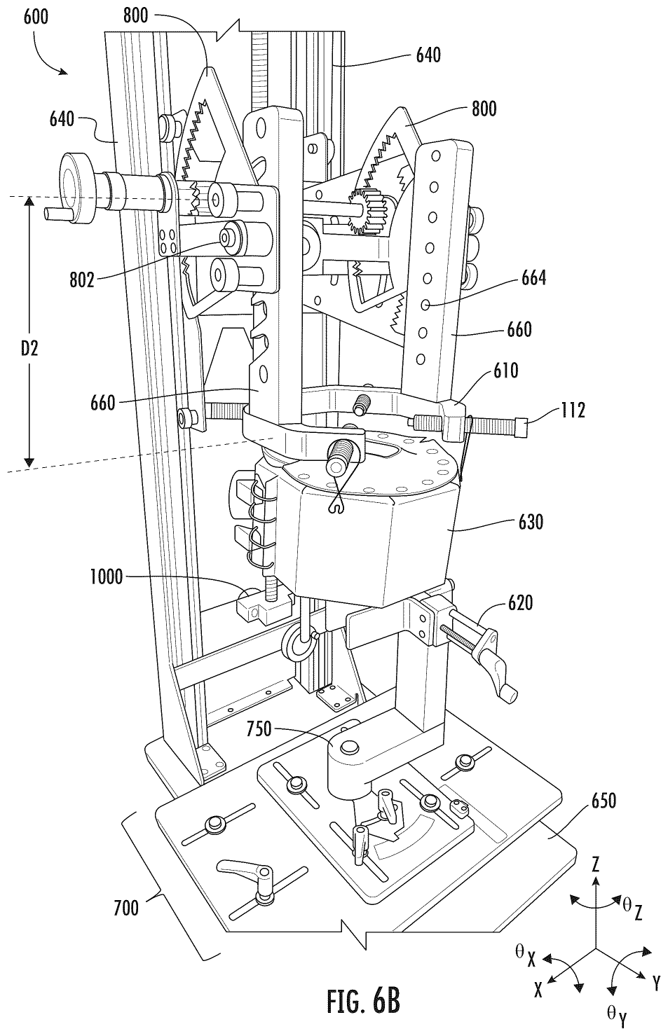

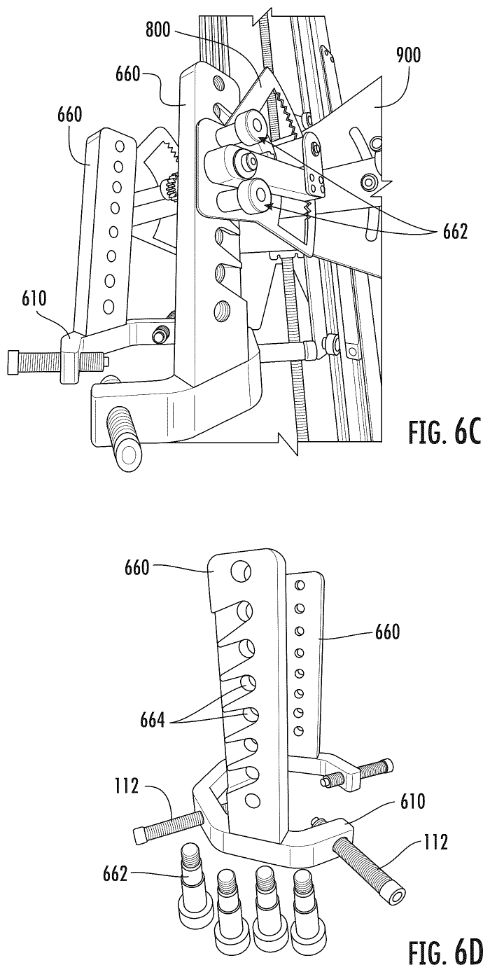

FIGS. 6A and 6B are perspective views of a thermoforming apparatus 600, in accordance with some aspects. Thermoforming apparatus 600 includes components for making adjustments to a medical device (e.g., medical device 10 of FIG. 1). As described for thermoforming apparatus 100, thermoforming apparatus 600 includes a first securing component 610 configured to hold a first region of the medical device (e.g., first region 22 of the medical device 10), and a second securing component 620 configured to hold a second region of the medical device (e.g., second region 24 of the medical device 10). Thermoforming apparatus 600 may include a heater 630 that is positioned between the first securing component 610 and the second securing component 620. The first securing component 610, second securing component 620, and heater 630 are mounted on a pair of vertical rails 640 in this example, with the rails 640 being supported by a base 650.

In some aspects, the thermoforming apparatus 600 may be used as a metrology and adjustment apparatus. For example, the heater 630 may be omitted or not turned on. In this manner, the apparatus can be used to measure alignment of components within the medical device 10 and/or make adjustments to the components, without utilizing the heater 630.

The thermoforming apparatus 600 is a fixture with adjustment mechanisms that enable multiple degrees of freedom in alignment of a medical device. The medical device may be a prosthesis or an orthosis for an upper extremity limb or a lower extremity limb as described throughout this disclosure. In some aspects, the thermoforming apparatus 600 includes a plurality of positioning mechanisms configured to provide i) translation in an X-direction, a Y-direction, and a Z-direction of a relative position of the first securing component and second securing component (and thereby the first region and the second region of the medical device) with respect to each other; ii) three degrees of freedom of rotation for the first region of the medical device, and iii) the three degrees of freedom of rotation for the second region of the medical device.

In some aspects, a metrology and adjustment apparatus for a medical device includes a first securing component configured to hold a first region of the medical device, and a second securing component configured to hold a second region of the medical device. The first securing component and the second securing component are spaced apart in a Z-direction. a plurality of positioning mechanisms configured to provide i) translation in an X-direction, a Y-direction, and the Z-direction for a relative position of the first securing component and the second securing component with respect to each other; and ii) at least two, such as three, degrees of freedom of angular orientation between the first securing component and the second securing component relative to each other. In some cases, a distance between the first securing component and a center of rotation of the first securing component is adjustable.

Each positioning mechanism enables adjustment of the medical device in a precise, quantifiable manner, in contrast to qualitative manual methods as in conventional practice. Some or all of the positioning mechanisms may include a scale, such as in millimeters for translation or degrees for rotation, to permit the user to monitor and measure the adjustments made in the various directions. The degrees of freedom include anterior and posterior directions (e.g., X-direction); superior and inferior (e.g., Z-direction); medial and lateral (e.g., Y-direction); and rotation about each of these axes (θ_{X} flexion and extension, θ_{Y} abduction and adduction, θ_{Z} medial and lateral rotation). FIG. 6B shows the first (upper) securing component 610 (and arms 660 coupled to the first securing component 610) being slightly tilted to the right compared to FIG. 6A, representing adjustment to a medical device that may occur during thermoforming.

The plurality of positioning mechanisms in thermoforming apparatus 600 includes a linear translation stage 700 on base 650 for making changes in the X and/or Y directions, a Z-rotation mechanism 750 for making changes in the θ_{Z} direction, an X-rotation mechanism 800 for making changes in the θ_{X} direction, a Y-rotation mechanism 900 (visible in FIG. 6A) for making changes in the θ_{Y} direction, and a Z-translation stage 1000 (partially visible in FIG. 6B) for making changes in the Z direction.

Also annotated in FIGS. 6A-6B are arms 660 that couple the Y-rotation mechanism 900 to the first securing component 610. The arms 660 are configured to allow adjustment of a distance “D1” between a Y-center of rotation 902 (FIG. 6A) of the Y-rotation mechanism 900 and the first securing component 610 (e.g., to the location of the holding elements 112). The arms 660 may also couple the X-rotation mechanism 800 to the first securing component 610 to enable adjustment of a distance “D2” between an X-center of rotation 802 (FIG. 6B) of the X-rotation mechanism 800 and the first securing component 610 (e.g., to the location of the holding elements 112). This unique adjustability provides even more customization in making patient-specific alignment adjustments to the medical devices, where the securing components may not be aligned to anatomical features such as knee/elbow joints, but where the center of rotation of adjustment must be at those anatomical features. In cases where the medical device is a transtibial prosthetic device, the ability to reposition the securing component allows for adjustable Patellar Tendon Bearing (PTB) height to maintain the center of rotation and accommodate prostheses with different length residua.

In this example, arms 660 have a plurality of holes 664 along their length that allow the first securing component 610 to be moved closer to or farther away from the Y-center of rotation 902. In other examples, the distance D1 may be adjusted by other mechanisms such as pins, rails, a linear stage, or the like. The adjustment increments may be discrete (e.g., with holes 664 spaced apart at certain intervals) or continuous (e.g., with a rail, track, or linear stage). In some examples, only one arm 660 may be utilized (e.g., on one side of or centered on the back of first securing component 610) instead of two arms 660 on opposites sides of the first securing component 610 as illustrated. In some examples, arms 660 may be used to couple the Y-rotation mechanism 900 to the second securing component 620 instead of or in addition to (e.g., using additional arms 660) the first securing component 610. In some examples, one or more arms 660 may be used to couple other positioning mechanisms of the thermoforming apparatus 600 to the first securing component 610 and/or the second securing component 620.

FIGS. 6C and 6D show an example scenario of a procedure for adjusting the distance of the first securing component 610 from rotation mechanisms. In FIG. 6C, shoulder bolts 662 are removed to unlock the arm 660. In this example, there are two bolts on each arm 660. FIG. 6D is a perspective view of the first securing component 610 (with attached arms 660) removed from the thermoforming apparatus 600, along with the shoulder bolts 662. The first securing component 610 is repositioned by positioning arm 660 at a different position with respect to X-rotation mechanism 800 and/or Y-rotation mechanism 900. The shoulder bolts 662 are then reinstalled into different holes 664 than previously installed, to lock the first securing component 610 at the selected distance D1/D2 (i.e., height level).

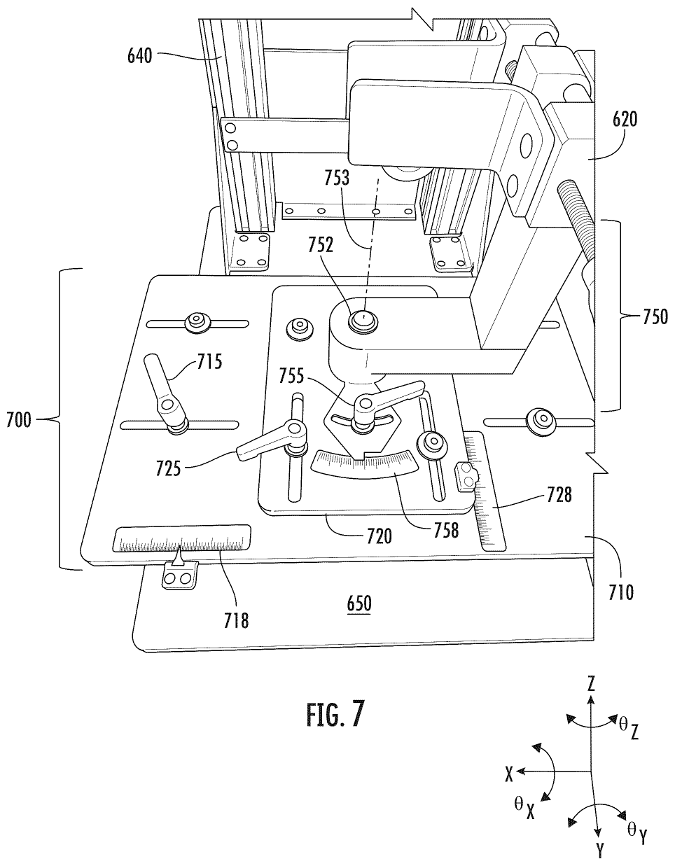

FIG. 7 is a perspective view of linear translation stage 700 and Z-rotation mechanism 750. The linear translation stage 700 is coupled to the second securing component 620 in this example (via Z-rotation mechanism 750). In other examples the linear translation stage 700 may be coupled to the first securing component 610 instead of to the second securing component 620, or a second linear translation stage 700 may be coupled to the first securing component 610 in addition to having a linear translation stage 700 for second securing component 620. Linear translation stage 700 is configured to provide translation in at least one of the X-direction or the Y-direction, such as by having pins that slide along slots oriented in the X- and/or Y-directions. In FIG. 7, the linear translation stage 700 is configured to move the second securing component 620 in both the X-direction and the Y-direction, where the Y-direction is perpendicular to the X-direction (both of which are perpendicular to the Z-direction). In this example, linear translation stage 700 comprises an X-stage 710 (i.e., translation table or plate) on base 650, and a separate Y-stage 720 (i.e., translation table or plate) that is on the X-stage 710. The X-stage 710 and Y-stage 720 each have slots in the X or Y direction, respectively, allowing translation in that direction. In other aspects, the Y-stage 720 may be underneath the X-stage 710 instead of on top. In other aspects, the linear translation stage 700 may be a single plate that includes both X and Y translation. The linear translation stage 700 enables translation in an X-direction and a Y-direction for a relative position of the second securing component 620 with respect to the first securing component 610, and therefore to adjust the relative positions of the first region 22 and the second region 24 of the medical device.

The X and Y movements of the linear translation stage 700 can be adjusted by hand, where each direction may have a locking handle to prevent the stages from moving when not in use. In the example illustrated in FIG. 7, the X-stage 710 has a locking handle 715, and the Y-stage 720 has a locking handle 725. The locks for both the X and Y translation directions set a fixed position for the second securing component 620, relative to which adjustments of the medical device are made (e.g., during thermoforming). Examples of mechanisms that may be used for locking handles 715 and 725 include cranks (using friction), detents, or fasteners such as pins or brackets. Each stage (X-stage 710 and Y-stage 720) also has a measurement scale, shown as X-scale 718 and Y-scale 728. In this illustration, the X-scale 718 for the X-axis and the Y-scale 728 for the Y-axis are physical scales (e.g., rulers or other scales read manually) and are both in millimeters. The range may be from, for example, −40 mm to +40 mm in this example. In other cases, the locking and/or measurement scales may be achieved by other techniques, such as through electronic locks, motors, pneumatics, hydraulics, optical detectors, digital readouts, electronic gages, or other types of position sensors. The measurement scales provide measurable values regarding the adjustments that are made using adjustments or thermoforming during a patient's fitting, which is important for monitoring patient conditions and for supplying documentation to insurance for reimbursement (e.g., that prosthesis adjustments are large enough that the need for a new prosthesis is warranted).

In one example, depending on how the medical device is mounted in the thermoforming apparatus 600, the X-direction (X-stage 710) may be for achieving anterior and posterior translation of the relative positions of the first region 22 and the second region 24, and the Y-direction may be for achieving medial and lateral translation of the relative positions of the first region 22 and the second region 24.

Also shown in FIG. 7 is Z-rotation mechanism 750 that is on top of the linear translation stage 700 and supports the second securing component 620. The Z-rotation mechanism 750 is configured to rotate about the Z-axis 753 that is perpendicular to a plane formed by the X-axis and the Y-axis. In the example of FIG. 7, the Z-rotation mechanism 750 includes an L-shaped arm that pivots around a Z-center of rotation 752 that has a Z-axis 753. The axis Z-axis 753 can, for example, be aligned with a central longitudinal axis of a medical device being adjusted. The second securing component 620 is attached to the upper portion of the L-shaped arm (see also FIGS. 5A-5B, second securing component 120). This rotation θ_{Z} (e.g., for toe-in, toe-out) transmits angular adjustments to the medical device via second securing component 620 in this example. In other examples, the Z-rotation mechanism 750 may be coupled to the first securing component 610. The Z-rotation mechanism 750 can be adjusted by hand and has a locking handle 755 in this example to prevent the Z-rotation mechanism 750 from moving when not in use (after being set in a desired position). In this illustration, the Oz scale 758 is in degrees, from −20° to +20°. As with X-scale 718 and Y-scale 728, the Oz scale 758 may include one or more of a physical scale, electronic locks, motors, pneumatics, hydraulics, optical detectors, digital readouts, electronic gages, or other types of angular sensors.

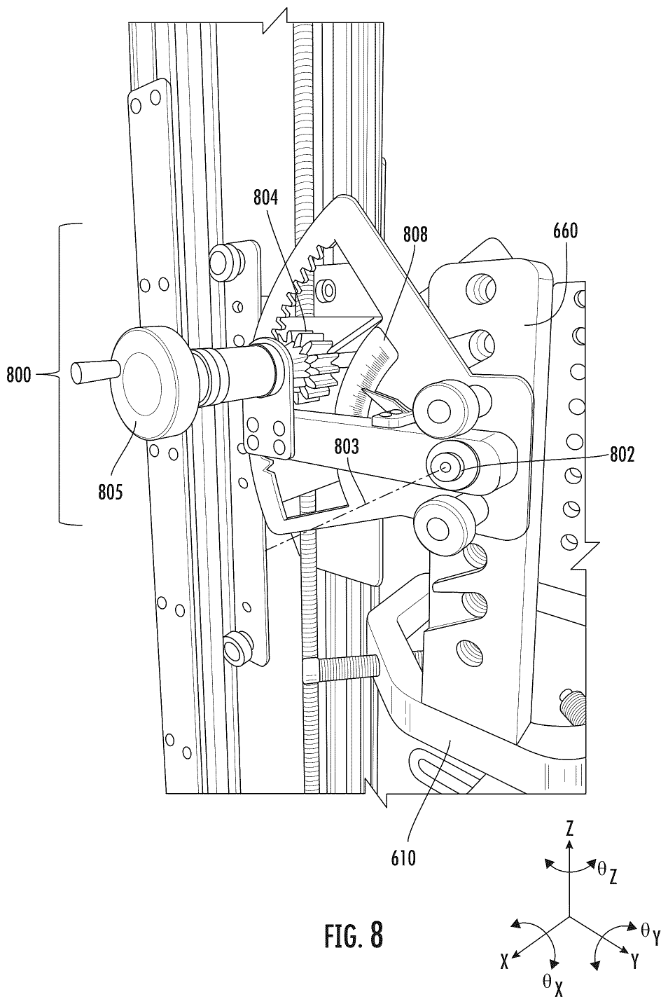

FIG. 8 shows a perspective view of X-rotation mechanism 800 for making alignment adjustments to the medical device in the Ox direction. In the example shown in FIGS. 6A-6B, two X-rotation mechanisms 800 are included, on the left and right sides of the thermoforming apparatus 600. In FIG. 8, the X-rotation mechanism 800 is coupled to the first securing component 610 and configured to rotate the first securing component about an X-center of rotation having an X-axis 803 that is oriented in the X-direction. In other cases, the X-rotation mechanism 800 may be coupled to the second securing component 620 instead of to the first securing component 610, or an additional X-rotation mechanism 800 may be coupled to the second securing component 620 in addition to having an X-rotation mechanism 800 for first securing component 610. The X-rotation mechanism 800 achieves the angular motion about X-center of rotation 802 through a gear mechanism 804 in this example, where alignments, adjustments or measurements are achieved through spinning the handle 805 (e.g., a crank or dial). The gear mechanism 804 may beneficially provide mechanical advantage, enabling a user to impart angular adjustments more easily and/or accurately than without a gear. In this example, handle 805 utilizes spring detents on the handle 805 to provide slip resistance and locking capability. The specific type of locking mechanism utilized (e.g., friction, detents, fasteners) may depend on the weight supported by the locking mechanism (e.g., weight of the arms 660 and/or securing component 610). X-rotation mechanism 800 also includes a measurement scale 808 for Ox in degrees, from −20° to +20° in this example. Measurement scale 808 may include one or more of a physical scale, electronic locks, motors, pneumatics, hydraulics, optical detectors, digital readouts, electronic gages, or other types of angular sensors.

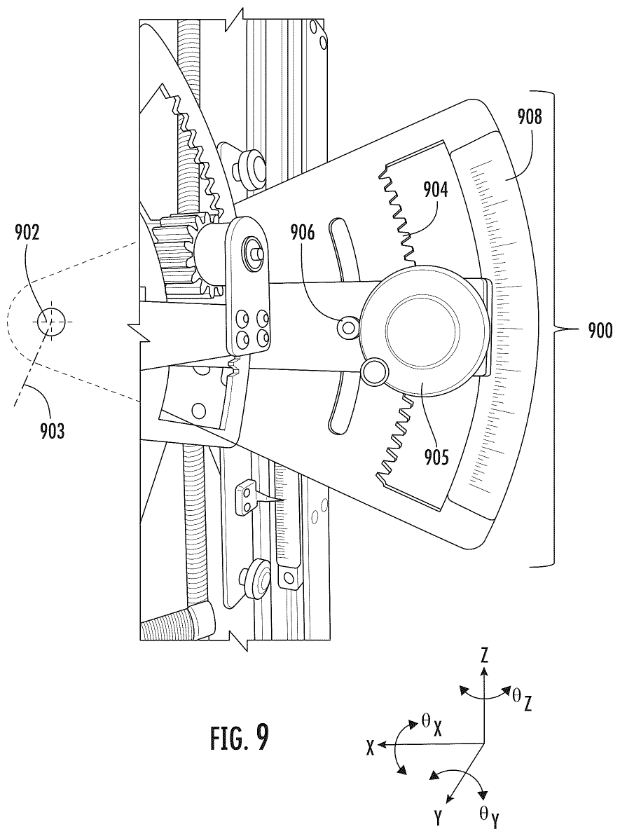

FIG. 9 shows a front view of Y-rotation mechanism 900 for making alignment adjustments to the medical device in the θ_{Y} direction. As shown in FIGS. 6A-6B the Y-rotation mechanism 900 is coupled to the first securing component 610 and configured to rotate the first securing component 610 about Y-center of rotation 902 that has a Y-axis 903 (FIG. 9) oriented in the Y-direction. In other examples, the Y-rotation mechanism 900 may be coupled to the second securing component 620 instead of to the first securing component 610, or an additional Y-rotation mechanism 900 may be coupled to the second securing component 620 in addition to having a Y-rotation mechanism 900 for first securing component 610. The Y-rotation mechanism 900 achieves the angular motion about the Y-center of rotation 902 through a gear mechanism 904 in this example. The gear mechanism 904 may beneficially provide mechanical advantage, enabling a user to impart angular adjustments more easily and/or accurately than without a gear. Adjustments, measurements or alignments of the medical device are achieved by turning the handle 905 (e.g., a crank or a dial). In the example of FIG. 9, a locking handle 906 is behind the handle 905, to lock the handle 905 by contacting the handle 905. In other aspects, other types of locking mechanisms may be used such as friction, detents, or fasteners. Y-rotation mechanism 900 also includes a measurement scale 908 for θ_{Y} in degrees, from −20° to +20° in this example. Measurement scale 908 may include one or more of a physical scale, electronic locks, motors, pneumatics, hydraulics, optical detectors, digital readouts, electronic gages, or other types of angular sensors.

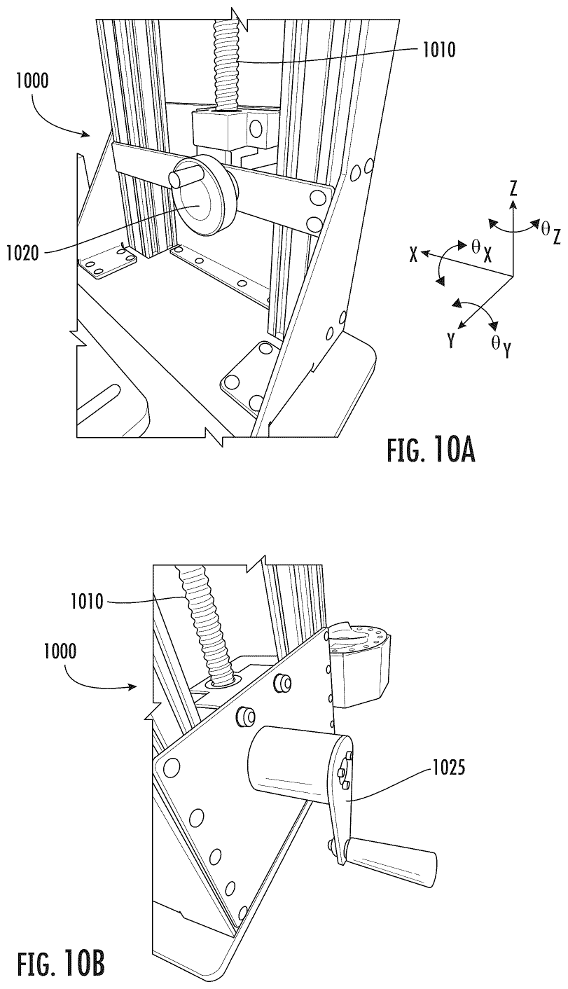

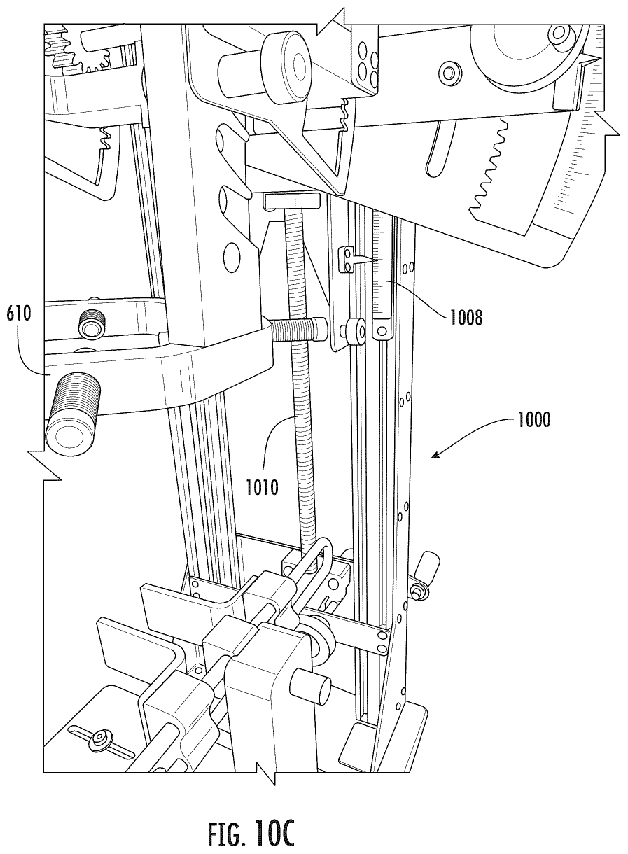

FIGS. 10A-10C show components of Z-translation stage 1000 for making Z-axis translation adjustments. FIG. 10A shows a front view, FIG. 10B shows a rear view, and FIG. 10C shows a top perspective view. The Z-translation stage 1000 is coupled to first securing component 610 (FIG. 10C), but in other examples may be coupled to second securing component 620. As can be seen in FIGS. 10A-10C, the Z-translation stage 1000 moves the first securing component 610 via a lead screw 1010. Actuation of the Z-translation stage 1000 lengthens or shortens the distance 116 (FIG. 2A) between the first securing component 610 and the second securing component 620. This Z-translation stage 1000 involves two handles in this example. A high torque, low speed handle 1020 is on the front side (FIG. 10A), and a low torque, high speed handle 1025 (FIG. 10B) is on the back of the thermoforming fixture. The high torque, low speed handle 1020 is for adjustments during thermoforming, where the high torque will stop the Z-translation stage 1000 from back driving. Consequently, no locking mechanism is required. However, in some aspects a locking mechanism may be included for the Z-translation stage 1000. The low torque, high speed handle 1025 may be used to make height adjustments (distance 116 between the first securing component 610 and the second securing component 620) during setup (i.e., loading the medical device into the thermoforming apparatus). A Z-axis scale 1008 is shown in FIG. 10C, with a scale of −40 mm to +40 mm in this example. The Z-axis scale 1008 is near the first securing component 610 in this example but may be positioned relative to the other components and moved and zeroed anywhere. Z-axis scale 1008 may include one or more of a physical scale (e.g., ruler), electronic locks, motors, pneumatics, hydraulics, optical detectors, digital readouts, or other types of position sensors.

There may be a maximum suggested amount of adjustment change in a single thermoforming cycle to avoid slippage of the first and second securing components 610 and 620 on the medical device. For example, a maximum suggested adjustment range in the Z-axis direction may be ±10 mm. The value can change depending on the types of clamps and holding components used in the securing components and can also depend on the ability of the medical device and thermoplastic material to be thermoformed (i.e., some materials and/or device configurations may be easier to thermoform than others).

Different configurations and combinations of the positioning mechanisms may be encompassed within the scope of this disclosure. For example, the Z-rotation mechanism 750 may be underneath the X-Y linear translation stage 700 instead of on top. In other examples, the X-rotation mechanism 800 and/or Y-rotation mechanism 900 may be coupled to the second securing component 620 (i.e., at the bottom of the thermoforming apparatus 600) instead of the first securing component 610 as shown in the figures. In general, the positioning mechanisms are configured above and below the heating zone. Furthermore, the combinations of the positioning mechanisms enable translational movement of the ends of the medical device relative to each other and rotation in three directions of the first region and second region of the medical device relative to each other. In some examples, one region of the medical device is fixed translationally while the other region can move translationally. The metrology and adjustment apparatus may enable rotational movements at each region (e.g., end) of the medical device, resulting in six degrees of freedom (involving translation and rotation). The plurality of positioning mechanisms may be configured to provide a combined total of at least six degrees of freedom between the first securing component and the second securing component, wherein the degrees of freedom comprise translational movement in an X-direction, a Y-direction, and the Z-direction and rotational movement about an X-axis, a Y-axis, and a Z-axis. In the example of a transtibial prosthetic limb, the foot and the socket may each be rotated in the three angular directions to provide proper alignment of those areas with respect to the pylon.

The actuation mechanisms (X, Y, Z translation and X, Y, Z rotation) may be configured to be actuated in various ways. For example, the positioning mechanisms (linear translation or rotational) may be actuated by manual force, manual or automated geared cranks, pneumatics (e.g., pneumatic motor or pneumatic piston), hydraulics (e.g., hydraulic piston), or electric motors.

The metrology and adjustment apparatus may also provide the ability to change the center of rotation of one or more of the angular adjustments to modify the location where bending or tilting will occur within the medical device during the alignment process. In cases where a heater is used such that the apparatus is a thermoforming apparatus, the location where bending or tilting will occur is in a heating zone (e.g., pylon 14). The modifying of the center of rotation location may be achieved, for example, by providing an adjustable distance between the securing component and center of rotation of a positioning mechanism, as explained above in relation to arms 660. The overall center of rotation for bending or tilting of a medical device when secured in the metrology and adjustment apparatus may be referred to as a centroid of axes of rotation or a centroid of rotation.

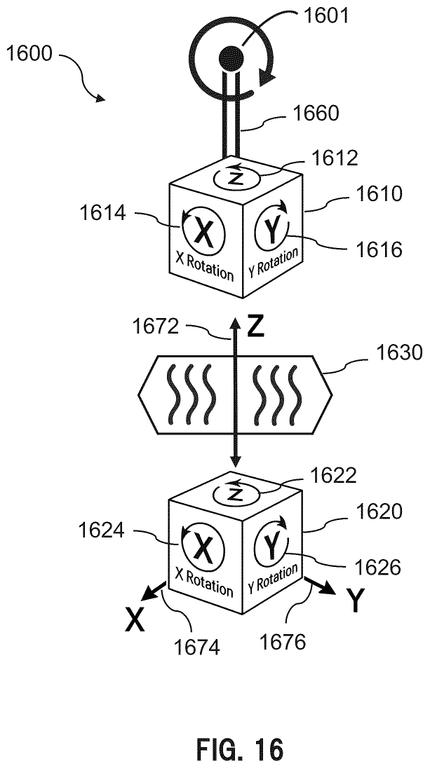

FIG. 16 is a schematic 1600 illustrating a centroid of axes of rotation 1601 for the metrology and adjustment apparatus, representing a point around which the medical device will bend (i.e., rotate) when held by the apparatus. FIG. 16 shows a first securing component 1610 (e.g., first securing component 110 or 610), a second securing component 1620 (e.g., second securing component 120 or 620), optional heater 1630 (e.g., heater 130 or 630), and arm 1660 (e.g., arm 660) coupling the first securing component 1610 to the centroid of axes of rotation 1601. Translational adjustments (e.g., linear) for the first securing component 1610 and/or the second securing component 1620 may be made in the Z-direction 1672, X-direction 1674 and/or Y-direction 1676. The first securing component 1610 and/or the second securing component 1620 each can rotate around individual centers of rotation around X, Y and/or Z axes, such as Z-rotation 1612, X-rotation 1614 and Y-rotation 1616 for first securing component 1610 and Z-rotation 1622, X-rotation 1624 and Y-rotation 1626 for second securing component 1620. The direction of rotation may be clockwise or counterclockwise.

The centroid of axes of rotation 1601 is a combined or composite center of rotation resulting from center of rotations for two or three of the X, Y, and Z axis, as well as the linear locations of the first securing component 1610 and second securing component 1620 (translational X, Y and Z distances relative to each other). For example, adjusting the translational and angular alignments of the first securing component 1610 and second securing component 1620 relative to each other will change the location of the centroid of axes of rotation 1601. First securing component 1610 is between centroid of axes of rotation 1601 and the second securing component 1620, for example in the Z-direction. Put another way, a center of rotation of the first securing component 1610 or the second securing component 1620 (i.e., center of rotation of one or both of the individual securing components) is offset from the centroid of axes of rotation 1601.

In one example, X-rotation 1614 may correspond to rotation about X-center of rotation 802 and X-axis 803 in FIG. 8. In another example, Y-rotation 1616 may correspond to rotation about Y-center of rotation 902 and Y-axis 903 in FIG. 9. In another example, Z-rotation 1622 may correspond to rotation about Z-center of rotation 752 and Z-axis 753 in FIG. 7. The overall centroid of axes of rotation 1601 is the location around which bending or tilting will occur when angular adjustments are made to a medical device secured in the metrology and adjustment apparatus. For instance, centroid of axes of rotation 1601 may be where X-axis 803 and Y-axis 903 (axes of rotation) intersect. In another example, centroid of axes of rotation 1601 may be where X-axis 803, Y-axis 903, and Z-axis 753 of rotation (axes of rotation) intersect.

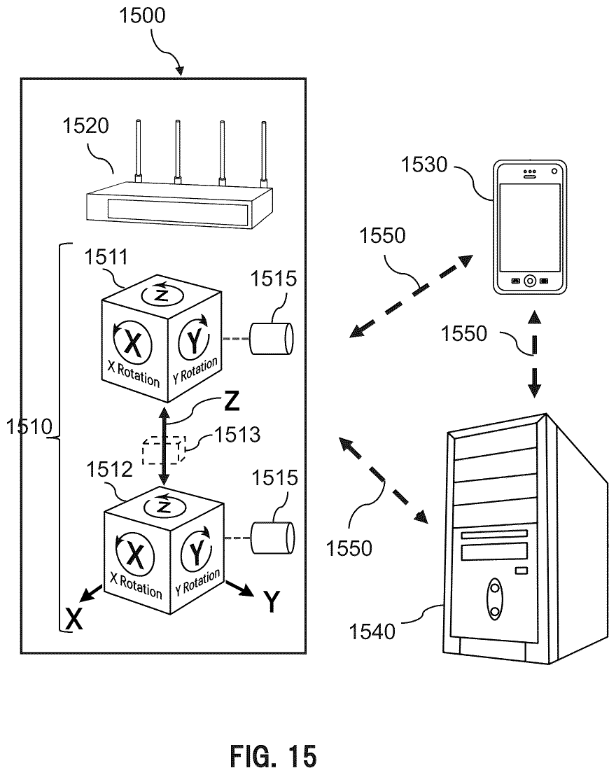

Additionally, the thermoforming apparatus of the present disclosure—which may also be a metrology and adjustment apparatus if used without operating or including the heater—advantageously provides measurement capability for the various adjustment parameters. In some aspects, at least one positioning mechanism of the plurality of positioning mechanisms comprises a measurement scale. Conventionally, the three-dimensional nature of aligning prostheses and orthoses is complex and difficult if not impossible to quantify using known techniques. In an example scenario, a medical device is mounted into the thermoforming apparatus, the user tares the initial settings (i.e., sets a starting position of zero or a recorded value), and the alignment adjustments are made. The resulting measurements can be recorded in the patient's records and reported in insurance to support reimbursement claims. These measurements may be read by the user, such as through visual scales or digital readouts, with the adjustments being made by the user. The adjustments may be made via the handles described herein, which may be in the form of cranks, dials, levers, or other actuation controls. In some aspects, at least one positioning mechanism of the plurality of positioning mechanisms comprises a lockable adjustment handle. In further aspects, automated adjustments may be made as shall be described in relation to FIG. 15, such as by typing in desired changes into a computer controller that is in communication with the positioning mechanisms. The controller then initiates the desired adjustments (e.g., set points) to the positioning mechanisms via electronic signals and automated (e.g., motorized) actuators.

In some aspects of the present disclosure, a metrology, adjustment, and/or thermoforming apparatus for a medical device comprises a first securing component configured to hold a first region of the medical device; and a second securing component configured to hold a second region of the medical device. A linear translation stage is coupled to the second securing component, the linear translation stage configured to move the second securing component in an X-direction and in a Y-direction that is perpendicular to the X-direction. A Z-translation stage is coupled to the first securing component, the Z-translation stage configured to move the first securing component in a Z-direction that is perpendicular to the X-direction and the Y-direction. An X-rotation mechanism is configured to rotate the first securing component about an X-center of rotation having an X-axis that is oriented in the X-direction. A Y-rotation mechanism is configured to rotate the first securing component about a Y-center of rotation having a Y-axis that is oriented in the Y-direction. A Z-rotation mechanism is configured to rotate the second securing component about a Z-axis that is perpendicular to the X-direction and the Y-direction. An arm couples the first securing component to at least one of the X-rotation mechanism or the Y-rotation mechanism, wherein the arm is configured to allow adjustment of a distance between the first securing component and at least one of the X-center of rotation and the Y-center of rotation. In some cases, a heater is positioned between the first securing component and the second securing component.

In some aspects of the present disclosure, a metrology, adjustment, and/or thermoforming apparatus for a medical device comprises a first securing component configured to hold a first region of the medical device. A second securing component is configured to hold a second region of the medical device, the first securing component and the second securing component being spaced apart in a Z-direction. A plurality of positioning mechanisms is configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other. A distance between the first securing component and a center of rotation of the first securing component is adjustable. In some cases, a heater is positioned between the first securing component and the second securing component.

In some aspects, A metrology and adjustment apparatus for a medical device, the apparatus comprising: a first securing component configured to hold a first region of the medical device; a second securing component configured to hold a second region of the medical device, the first securing component and the second securing component being spaced apart in a Z-direction; a plurality of positioning mechanisms coupled to at least one of the first securing component or the second securing component, the plurality of positioning mechanisms being configured to provide a combined total of at least six degrees of freedom between the first securing component and the second securing component, wherein the six degrees of freedom comprise translational movement in an X-direction, a Y-direction, and the Z-direction and rotational movement about an X-axis, a Y-axis, and a Z-axis; and a heater positioned between the first securing component and the second securing component.

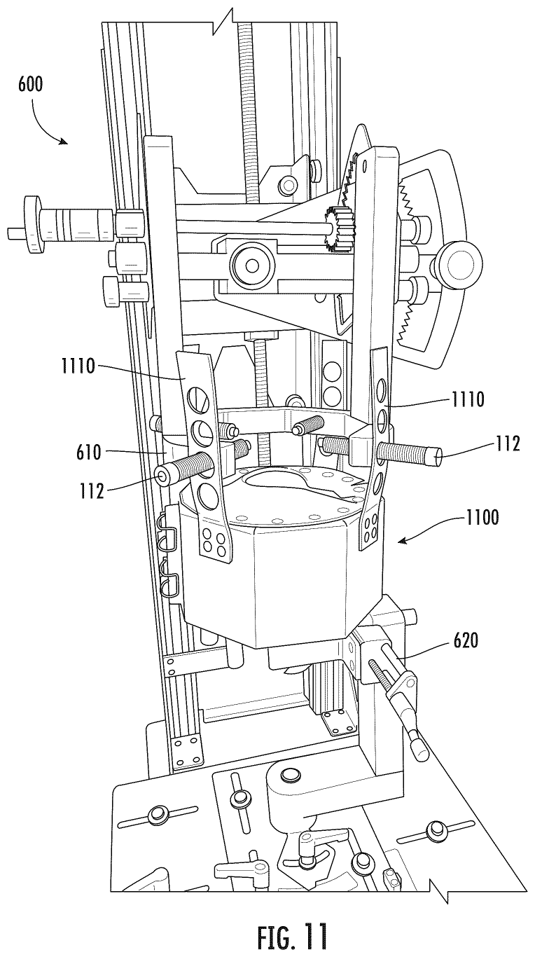

Turning to FIG. 11, a front view of a heater 1100 is shown in thermoforming apparatus 600. As described above, heater 1100 is positioned between the first securing component 610 and the second securing component 620 and provides heat to raise the temperature of the heating zone of the medical device (e.g., prosthesis or orthosis) to a point of pliability. The heater 1100 may be removable from the overall thermoforming fixture to facilitate installment of the medical device into the apparatus. In this example of FIG. 11, the heater 1100 has straps 1110 that enable the heater 1100 to be suspended or removed from the holding elements 112 of the first securing component. In FIG. 11, the straps 1110 have multiple mounting holes to change the location of heater 1100 between first securing component 610 and second securing component 620, accommodate various positions of the desired heat zones due to varying sizes (e.g., lengths) of the prostheses or orthoses.

The heater 1100 is configured as a chamber or enclosure that contains heat so that the medical device can be brought to the necessary temperature in an efficient manner. When mounted in the thermoforming apparatus, the medical device extends through the center of the heater 1100 such that the region to be heated (e.g., pylon 14) is surrounded by the heater 1100. The temperature of the heated zone (e.g., pylon 14) is raised until it is pliable; that is, above the material's glass transition temperature and below the material's melting temperature. Aspects include monitoring the temperature of the air in the heater 1100 and/or the heated material of the medical device itself using one or more temperature sensors such as a thermocouple or an optical sensor.

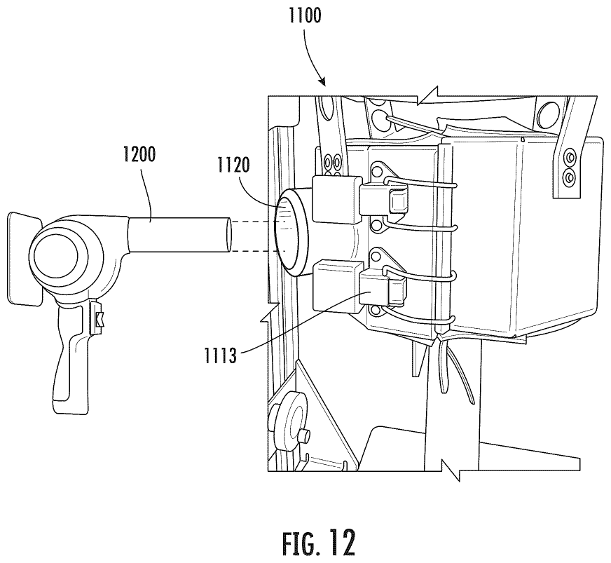

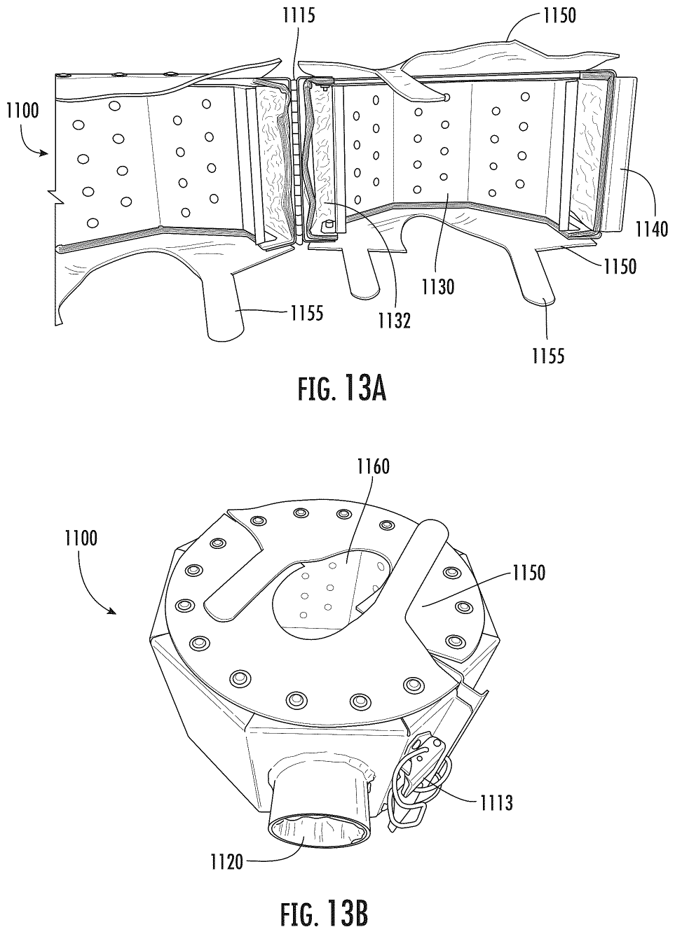

FIGS. 12 and 13A-13B describe an example of heater 1100 that utilizes a heat gun as a heat source 1200. The heat gun is an example of an electric heater (e.g., a resistance heater). In other examples, other sources for generating heat may be used, such as infrared heating, resistance heating, radiative heating, convective heating, or inductive heating. Convective heating may use fluids or gases such as air, water, or any other thermally conductive medium. The heater 1100 may be configured to evenly provide heating around the medical device. In some cases, the heater 1100 comprises a chamber having lateral walls configured to emanate heat from a perimeter of the chamber (i.e., enclosure) into an interior of the chamber. The heat source 1200 may be separate from the chamber or may be integrated into the chamber (e.g., heating elements mounted on exterior or interior walls of the chamber). In the example of FIG. 12, the heat source 1200 is an electric heater that is external to (e.g., separate from) the chamber and is used to heat a fluid (e.g., air) that is used to convey thermal energy to the medical device via convective heat transfer. The heat source 1200 may be configured to achieve a temperature needed to thermoform the material of the medical device that is being adjusted.

In the side view of FIG. 12, the heat source 1200 may be coupled to the heater 1100 at a port 1120 of the heater 1100. In this example, the chamber of the heater 1100 has clasps 1113 that secure together two halves of the chamber that are attached by hinges 1115, as shown in FIG. 13A. In other examples, the chamber can be made of three or more pieces, or may be a one-piece sleeve that is slid over the medical device before mounting it into the thermoforming apparatus.

FIG. 13A is a view of the heater 1100 in which the two halves are open. The heater 1100 may be a thermal chamber that distributes hot air around the medical device, such as being configured as a diffuser. Convective heating can promote uniform heating around the medical device. In this example, air from the heat gun (heat source 1200) flows through a channel 1132 formed by an inner wall 1130 and an outer wall 1140 of the heater 1100. The inner wall 1130 or outer wall 1140 are lateral walls of a perimeter of the chamber. Holes in the inner wall 1130 allow air to heat (or cool, if cool air is blown from the heat gun) the medical device that is inside the chamber. The holes may be distributed across the width and height of the inner wall 1130. In this manner, the chamber emanates heat from a perimeter of the chamber and provides uniform heating around the circumference and the length of the portion of the medical device that is inside the heater 1100. Uniform heating within the chamber helps ensure that the medical device can be adjusted in any direction desired.

FIGS. 13A-13B show a cover 1150 that may be attached to the top and bottom of the heater 1100 in some cases. Cover 1150 may be made of a flexible and heat-tolerant material that allows medical devices of various cross-sectional shapes and sizes to be enclosed within the heater 1100. The cover 1150 is configured to form an aperture 1160 (FIG. 13B) that is adjustable in size by flaps 1155 of the cover 1150. By using a flexible material for the cover 1150, the cover 1150 can conform around the medical device to help prevent heat from escaping from the heater 1100. In one example, the cover 1150 may be made of silicone.

In some aspects, heater 1100 may be configured to apply heat to the heating zone of the medical device without fully enclosing the heating zone. For example, the walls of the heater 1100 may have gaps or be discontinuous (e.g., separate plates arranged around the perimeter of the medical device) while still applying sufficient heat to cause thermoforming. In general aspects, the heating zone of the medical device may be positioned within the heater 1100, where the heating zone may be fully or partially surrounded by the heater.

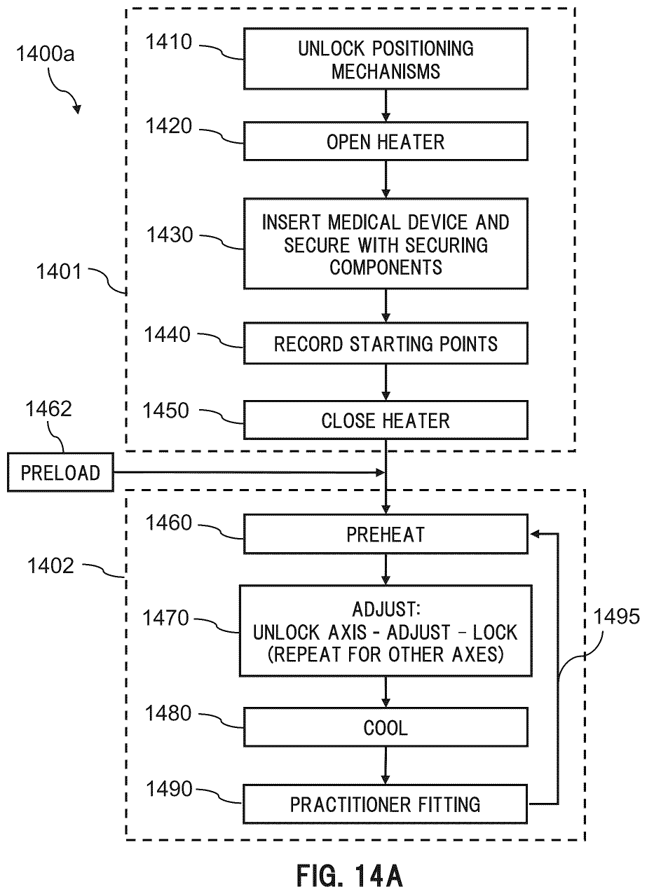

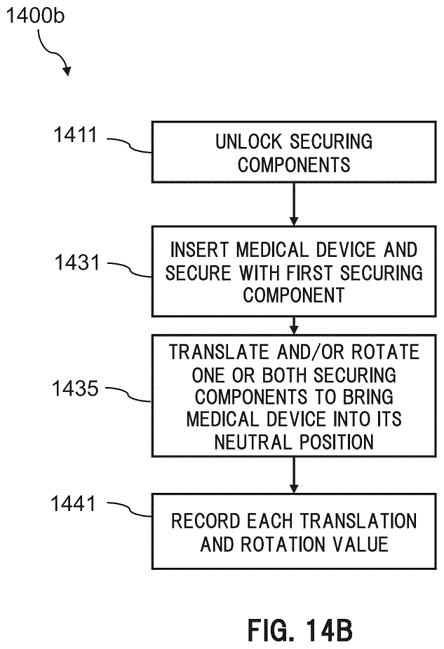

FIG. 14A is a flowchart representing a method 1400a for measuring, adjusting, and/or thermoforming a medical device, in accordance with aspects of the present disclosure. The medical device may be, for example, a prosthesis or an orthosis, such as for a leg arm, or other body part. The medical device may be thermoformable (e.g., made with a material that becomes pliable when heated) or not thermoformable. The method 1400a includes setup phase 1401 and adjusting phase 1402.

During setup phase 1401, a metrology and adjustment apparatus as described herein is provided in block 1410, where the apparatus has a first securing component and a second securing component that are spaced apart in a Z-direction. In block 1410 all positioning mechanisms of the securing components that have locks can be unlocked (e.g., X, Y, Z translation, and/or X, Y, Z-rotation) if they are not already unlocked. One or more of the measurement scales on the positioning mechanisms (e.g., as described in FIGS. 7, 8, 9, 10C) may be zeroed or tared. For example, the rotation scales θ_{X}, θ_{Y} and/or θ_{Z} may be set to zero degrees, or the starting value may be noted (e.g., recorded, stored). For medical devices which will be thermoformed, the heater (i.e., thermal chamber) is opened in optional block 1420. The medical device is inserted into the metrology and adjustment apparatus (e.g., the apparatuses 100 or 600) in block 1430. In block 1430, the medical device is secured to the first securing component and the second securing component, and the portion of the medical device that is to be measured, adjusted and/or thermoformed is placed into the heater. In some aspects, alignment fixture datums (alignment features 310) are used to secure the medical device (e.g., prosthetic device). During the securing, the Z-translation mechanism may be actuated (e.g., using handle 1025) to move the first securing feature to the location of the first region 22 (e.g., socket-pylon joint for a transtibial prosthesis). In other aspects, the second securing feature (lower clamp) is moved by the Z-translation mechanism to be positioned at the location of the second region 24 (e.g., ankle-foot joint for a transtibial prosthesis). For a transtibial device, the ankle gap should be just below the center of the lower clamp. After the medical device is secured, the starting points of the various positioning mechanisms are recorded in block 1440. If being used, the thermal chamber is closed in block 1450, and thermoforming is ready to begin. If thermoforming is not being performed, block 1450 may be omitted, and the method proceeds from block 1440 to the adjusting phase 1402.

In one example method of the setup phase 1401, after the correct Z-height is set for the upper positioning mechanism (e.g., first securing component 610), the mechanism is tightened. The lower clamp (e.g., second securing component 620) may be designed to be self-centering, and the X-Y translation tables are allowed to “float” to their starting points. The rotation values for X, Y, and Z (θ_{X}, θ_{Y}, θ_{Z}) are then locked, and the measurements for the starting values are recorded in block 1440.

To thermoform the medical device in the adjusting phase 1402 of method 1400a, the heat source (e.g., heat gun) is turned on to begin preheating the medical device in block 1460. A time limit for a heating cycle may be set to ensure the medical device is not overheated, thus preventing damage to the medical device. In one example, the medical device may be heated (during one thermoforming cycle) for a maximum of 10 minutes, which allows 5 minutes for preheating and 5 minutes for forming. The heating time and temperature will depend on the specific material and design of the medical device. The initial heating period (preheating, e.g., the first 5 minutes) allows the medical device to absorb enough heat to become pliable and thereby take on alignment modifications. During an adjustment that involves thermoforming, in adjusting phase 1402 the practitioner unlocks the axis that is desired to be adjusted, which can be translation or rotation. All other axes should be locked so that they cannot move. The user actuates the positioning mechanism (e.g., by turning a handle, starting a motorized actuator) to make alignments as necessary (e.g., based on the patient's deviations presented, such as by gait analysis). After alignments are made in a particular axis, that axis is locked. Adjustments for other axes can then be performed; that is, multiple alignments may be performed in one heat cycle. The thermoforming steps of unlocking an axis, making alignment adjustments, and locking the axis is repeated for other axes (positioning mechanisms) during block 1470 as needed and as time allows. The adjusted positions/angles of the mechanisms may also be recorded.

When the alignments for that heat cycle are completed, or when the time limit for the heat cycle is up, the heat source is turned off. The medical device is then cooled during block 1480. During cooling, various techniques can be taken to help cool the medical device. In an example of a heat gun as the heat source, the heat gun may continue blowing air but with the heating turned off so that the air can provide convective cooling of the prosthetic device. In general examples of achieving cooling, the thermal chamber can be opened, an external fan may be used, and/or a damp towel or mist may be applied on the medical device. Cooling is necessary before removing the medical device from the thermoforming apparatus to set the adjustment changes within the thermoplastic material. Temperature measurements may be taken during the forming phase, such as to monitor the temperature within the heater or the temperature of the heated area of the medical device itself. Temperature measurements may help determine when the medical device is ready for adjustments to be made or is cool enough to remove from the apparatus.

In another aspect, adjustment of the medical device may be achieved using a preloading force imposed on the medical device prior to thermoforming, in block 1462 before preheating the medical device in block 1460. For example, a certain amount of force may be applied via the first and/or second securing components in one or more translational or rotational directions prior to heating. The preloading force(s) may be applied by moving one or more of the positioning mechanisms for the first and/or second securing components. Then when the medical device is heated in block 1460, the loads will cause alignment changes, where the preloaded stresses will become relaxed during heating. In this preloading scenario, block 1470 of adjusting the medical device occurs due to the preloading forces. The medical device is then cooled in block 1480.

After thermoforming, the adjusted medical device is ready for fitting by a medical practitioner in optional block 1490, such as a prosthetist or orthotist. The thermoforming can be repeated after the practitioner fitting, as indicated by loop 1495, to ensure proper alignment and fitment.

FIG. 14B shows a method 1400b in which the metrology and adjustment apparatus is used for measuring and/or aligning a medical device without thermoforming, in accordance with some aspects. In these cases, block 1420 (opening the heater), block 1450 (closing the heater), block 1460 (preheating) and block 1480 (cooling) of FIG. 14B may be omitted. As an example, method 1400b may be used for a transtibial prosthetic device having a socket, a pipe as a pylon, and a foot.

A metrology workflow using method 1400b shall be described using the replacement of a socket for a transtibial prosthesis without losing alignment, as an example. In block 1411, securing components are unlocked. In block 1431, the medical device is inserted into the apparatus and secured with the first securing component. In block 1435, the medical device is adjusted using a plurality of positioning mechanisms of the metrology and adjustment apparatus. One or both of the first and second securing components are translated and/or rotated as needed to bring the medical device into its neutral position. For instance, the existing prosthesis with first socket may be loaded into the apparatus, and its alignment measured. Neutral position may be set by the practitioner, such as by having the pylon vertical (along the Z-axis), with the foot flat and the medial border of foot being aligned along the X-axis. Measurement can be made by reading each translation and rotation axis, or by taking a 3D scan of the device within the apparatus. These values are recorded in block 1441 to establish the alignment (i.e., neutral position) of the existing (original) prothesis components. The prosthesis is removed, and the socket is detached from the pylon-ankle-foot assembly. The pylon-ankle-foot is loaded back into the apparatus. The replacement socket is loaded into the apparatus, and one or both securing components are translated and/or rotated using the positioning mechanisms described herein to match the correct alignment as recorded in block 1441. The replacement socket is then attached to the prosthesis in the correct aligned position as set by the apparatus. The prosthesis with its socket replaced can then be removed from the apparatus.

In some aspects, a method of adjusting a medical device includes providing a metrology and adjustment apparatus having a first securing component, a second securing component, and a heater between the first securing component and the second securing component, wherein the first securing component and the second securing component are spaced apart in a Z-direction. The method also includes securing a first region of the medical device with the first securing component; securing a second region of the medical device with the second securing component; positioning a heating zone of the medical device within the heater; heating the heating zone of the medical device. The method also includes adjusting a plurality of positioning mechanisms coupled to the first securing component and the second securing component to thermoform the medical device, wherein the plurality of positioning mechanisms are configured to provide: i) translation in an X-direction, a Y-direction, and the Z-direction for a relative position of the first securing component and the second securing component with respect to each other; and ii) at least two degrees of freedom of angular orientation between the first securing component and the second securing component relative to each other.

In some aspects, a method of adjusting a medical device includes a) providing a metrology and adjustment apparatus having a first securing component, a second securing component, and a heater between the first securing component and the second securing component, wherein the first securing component and the second securing component are spaced apart in a Z-direction. The method also includes securing a first region of the medical device with the first securing component; securing a second region of the medical device with the second securing component; positioning a heating zone of the medical device within the heater; heating the heating zone of the medical device. The method also includes adjusting a plurality of positioning mechanisms coupled to the first securing component and the second securing component to thermoform the medical device, wherein the plurality of positioning mechanisms are configured to provide: i) translation in an X-direction, a Y-direction, and the Z-direction for a relative position of the first securing component and the second securing component with respect to each other; and ii) at least two degrees of freedom of angular orientation between the first securing component and the second securing component relative to each other. The method also includes b) securing a first region of the medical device with the first securing component; c) securing a second region of the medical device with the second securing component; and d) adjusting the medical device using the plurality of positioning mechanisms.

FIG. 15 is a block diagram of a metrology and adjustment system 1500 that includes automated control and reporting capabilities, in accordance with some aspects. The metrology and adjustment apparatus 1510 (e.g., apparatus 100 or 600) is in communication with a networking and control system 1520 that is configured to actuate the plurality of positioning mechanisms of the metrology and adjustment apparatus 1510. Metrology and adjustment apparatus 1510 includes first securing component 1511, second securing component 1512, and optional heater 1513. The networking and control system 1520 can connect to an internet or mobile telecommunication network by connections 1550, which may be hard-wired or wireless. A data collection device 1530 (e.g., smartphone, computer tablet, laptop, or other electronic device) is used to capture, enter, and/or transmit information from the patient, the information including but not limited to videos, gait analysis, alignment values, biomechanical forces, patient height, weight, and 3D scans. A computer processor 1540 serves as a storage and computation system which may be used to convert the information (i.e., data) from data collection device 1530 into machine commands, such as G-code, that networking and control system 1520 uses to actuate various translations, rotations, or heating in metrology and adjustment apparatus 1510. The translations and rotations of components in metrology and adjustment apparatus 1510 may be actuated by a drive mechanism 1515 which may be, for example, a motor (e.g., electric, pneumatic, hydraulic), pistons (e.g., electric, pneumatic, hydraulic), gears, cranks, or other actuators, A graphical user interface (e.g., visual monitor or display) and/or input device (e.g., keyboard, touch screen or other) may be connected to metrology and adjustment system 1500 or computer processor 1540 for data entry and control of the metrology and adjustment system 1500. Any commands may be reported between data collection device 1530 and computer processor 1540, metrology and adjustment system 1500 and computer processor 1540, or metrology and adjustment system 1500 and data collection device 1530 so that the measurements and adjustments are captured.

In one example, a clinician may use data collection device 1530 to take a video of a patient walking. Data collection device 1530 may be, for example, a laptop, a computer table, a smartphone, or other electronic device. The data is transmitted to computer processor 1540 where the gait is analyzed and alignment adjustments are determined, and these adjustments are transmitted to networking and control system 1520 where the correct translations, rotations, and heating are executed through motors, heaters, and sensors to automatically thermoform a prosthetic leg. The total alignment adjustment in all degrees of freedom is then transmitted to computer processor 1540 where a generated report may be sent to insurance for medical justification purposes. Aspects of metrology and adjustment system 1500 include a human-machine interface via data collection device 1530 and connection 1550, remote control of the metrology and adjustment apparatus 1510, data uploading, and data export.

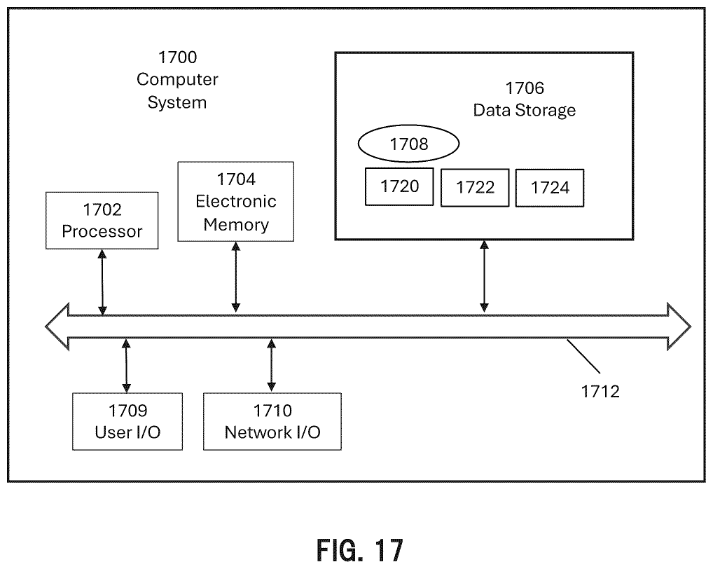

FIG. 17 is a simplified schematic diagram showing an example computer system 1700 (representing any combination of one or more of the computer systems) for use in the methods and systems of the present disclosure. The computer system 1700 may be, for example, networking and control system 1520 or computer processor 1540. Computer system 1700 represents one or more computer processors that may be used to perform various steps of the methods described herein, such as for storing measurements and/or computing, and implementing adjustments (e.g., alignments) for a medical device using the metrology and adjustment apparatuses described herein. These various steps may be all be performed by one computer processor, or some steps may be performed by one computer processor while other steps are performed by another computer processor. The computer processor may be part of, for example, a smartphone, a computer tablet, or a computer workstation (e.g., desktop computer or laptop/notebook computer) that is used by a prosthetist or other professional working with the medical device (e.g., prosthesis or orthosis).

In the illustrated example, the computer system 1700 generally includes at least one processor 1702, at least one main electronic memory 1704, at least one data storage 1706, at least one user I/O 1709, and at least one network I/O 1710, among other components not shown for simplicity, connected or coupled together by a data communication subsystem 1712.

The processor 1702 represents one or more central processing units on one or more PCBs (printed circuit boards) in one or more housings or enclosures. In some examples, the processor 1702 represents multiple microprocessor units in multiple computer devices at multiple physical locations interconnected by one or more data channels. When executing computer-executable instructions for performing the above-described functions of the computer system 1700 in cooperation with the main electronic memory 1704, the processor 1702 becomes a special purpose computer for performing the functions of the instructions.

The main electronic memory 1704 represents one or more RAM modules on one or more PCBs in one or more housings or enclosures. In some cases, the main electronic memory 1704 represents multiple memory module units in multiple computer devices at multiple physical locations, which may include cloud servers or cloud storage.

The data storage 1706 represents or comprises any appropriate number or combination of internal or external physical mass storage devices, such as hard drives, optical drives, network-attached storage (NAS) devices, flash drives, etc. In some cases, the data storage 1706 represents multiple mass storage devices in multiple computer devices at multiple physical locations which may include cloud storage. The data storage 1706 generally provides persistent storage (e.g., in a non-transitory computer-readable or machine-readable medium 1708) for the programs (e.g., computer-executable instructions) and data used in operation of the processor 1702 and the main electronic memory 1704. The non-transitory computer readable medium 1708 includes instructions (e.g., the programs and data 1720, 1722, 1724) that, when executed by the processor 1702, cause the processor 1702 to perform operations including the above-described functions of the computer system 1700.

In some examples, the main electronic memory 1704 and the data storage 1706 include all, or a portion of the programs and data (e.g., represented by 1720-1748) required by the processor 1702 to perform the methods, processes and functions disclosed herein (e.g., in FIGS. 4-6). Under control of these programs and using this data, the processor 1702, in cooperation with the main electronic memory 1704, performs the above-described functions for the computer system 1700.

The user I/O 1709 represents one or more appropriate user interface devices, such as keyboards, pointing devices, displays, etc. In some examples, the user I/O 1709 represents multiple user interface devices for multiple computer devices at multiple physical locations. A system administrator, for example, may use these devices to access, set up, and control the computer system 1700.

The network I/O 1710 represents any appropriate networking devices, such as network adapters, etc., for communicating throughout the system. In some examples, the network I/O 1710 represents multiple such networking devices for multiple computer devices at multiple physical locations for communicating through multiple data channels.

The data communication subsystem 1712 represents any appropriate communication hardware for connecting the other components in a single unit or in a distributed manner on one or more PCBs, within one or more housings or enclosures, within one or more rack assemblies, within one or more geographical locations, etc.

Example aspects of the present systems and methods are described in the clauses below.

Clause 1. A metrology and adjustment apparatus for a medical device, the apparatus comprising: a first securing component configured to hold a first region of the medical device; a second securing component configured to hold a second region of the medical device; a linear translation stage coupled to the second securing component, the linear translation stage configured to move the second securing component in an X-direction and in a Y-direction that is perpendicular to the X-direction; a Z-rotation mechanism configured to rotate the second securing component about a Z-axis that is perpendicular to the X-direction and the Y-direction; a Z-translation stage coupled to the first securing component, the Z-translation stage configured to move the first securing component in a Z-direction that is perpendicular to the X-direction and the Y-direction; an X-rotation mechanism configured to rotate the first securing component about an X-center of rotation having an X-axis that is oriented in the X-direction; a Y-rotation mechanism configured to rotate the first securing component about a Y-center of rotation having a Y-axis that is oriented in the Y-direction; and an arm that couples the first securing component to at least one of the X-rotation mechanism or the Y-rotation mechanism, wherein the arm is configured to allow adjustment of a distance between the first securing component and at least one of the X-center of rotation and the Y-center of rotation.

Clause 2. The metrology and adjustment apparatus of clause 1, wherein the medical device is a prosthesis or an orthosis for a leg.

Clause 3. The metrology and adjustment apparatus of any of clauses 1-2, wherein the Z-translation stage is mounted on a pair of rails.

Clause 4. The metrology and adjustment apparatus of any of clauses 1-3, wherein the X-center of rotation or the Y-center of rotation is offset from a centroid of axes of rotation.

Clause 5. The metrology and adjustment apparatus of any of clauses 1-4, wherein the linear translation stage, the Z-translation stage, the Z-rotation mechanism, the X-rotation mechanism, or the Y-rotation mechanism comprises a measurement scale.

Clause 6. The metrology and adjustment apparatus of clause 5, wherein the measurement scale comprises an electronic gage.

Clause 7. The metrology and adjustment apparatus of any of clauses 1-6, wherein the linear translation stage, the Z-translation stage, the Z-rotation mechanism, the X-rotation mechanism, or the Y-rotation mechanism comprises a lockable adjustment handle.

Clause 8. The metrology and adjustment apparatus of any of clauses 1-7, wherein the linear translation stage, the Z-translation stage, the Z-rotation mechanism, the X-rotation mechanism, or the Y-rotation mechanism comprises a pneumatic motor or an electric motor.

Clause 9. The metrology and adjustment apparatus of any of clauses 1-8, further comprising a heater positioned between the first securing component and the second securing component.

Clause 10. The metrology and adjustment apparatus of clause 9, wherein the heater comprises a channel formed by lateral walls of a perimeter of a chamber of the heater.

Clause 11. The metrology and adjustment apparatus of clause 9, wherein the heater comprises an electric heater integrated within a chamber of the heater.

Clause 12. The metrology and adjustment apparatus of clause 9, wherein the heater comprises an electric heater that is external to a chamber of the heater and is used to heat a fluid to heat the medical device via convective heat transfer.

Clause 13. A metrology and adjustment apparatus for a medical device, the apparatus comprising: a first securing component configured to hold a first region of the medical device; a second securing component configured to hold a second region of the medical device, the first securing component and the second securing component being spaced apart in a Z-direction; and a plurality of positioning mechanisms configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other; wherein a distance between the first securing component and a center of rotation of the first securing component is adjustable.

Clause 14. The metrology and adjustment apparatus of clause 13, wherein the medical device is a prosthesis or an orthosis for a leg.

Clause 15. The metrology and adjustment apparatus of any of clauses 13-14, wherein the plurality of positioning mechanisms comprises a linear translation stage coupled to the first securing component or the second securing component, the linear translation stage configured to provide the translation in the X-direction or the Y-direction.

Clause 16. The metrology and adjustment apparatus of any of clauses 13-15, wherein the plurality of positioning mechanisms comprises a Z-translation stage coupled to the first securing component or the second securing component, the Z-translation stage configured to provide the translation in the Z-direction.

Clause 17. The metrology and adjustment apparatus of clause 16, wherein the Z-translation stage is mounted on a pair of rails.

Clause 18. The metrology and adjustment apparatus of any of clauses 13-17, wherein: the plurality of positioning mechanisms comprises an X-rotation mechanism, a Y-rotation mechanism, and a Z-rotation mechanism configured to achieve three degrees of freedom of rotation of the first region or the second region; the X-rotation mechanism is configured to rotate the first securing component or the second securing component about an X-axis that is oriented in the X-direction; the Y-rotation mechanism is configured to rotate the first securing component or the second securing component about a Y-axis that is oriented in the Y-direction; and the Z-rotation mechanism is configured to rotate the first securing component or the second securing component about a Z-axis that is perpendicular to a plane formed by the X-axis and the Y-axis.

Clause 19. The metrology and adjustment apparatus of clause 18, further comprising an arm that couples the X-rotation mechanism or the Y-rotation mechanism to the first securing component, wherein the arm is configured to allow adjustment of the distance between the first securing component and the center of rotation.

Clause 20. The metrology and adjustment apparatus of clause 19, wherein the arm comprises a plurality of holes along a length of the arm.

Clause 21. The metrology and adjustment apparatus of clause 19, wherein the center of rotation is an X-center of rotation of the X-rotation mechanism or a Y-center of rotation of the Y-rotation mechanism.

Clause 22. The metrology and adjustment apparatus of clause 21, wherein the X-center of rotation or the Y-center of rotation is offset from a centroid of axes of rotation.

Clause 23. The metrology and adjustment apparatus of any of clauses 13-22, wherein a positioning mechanism of the plurality of positioning mechanisms comprises a measurement scale.

Clause 24. The metrology and adjustment apparatus of clause 23, wherein the measurement scale comprises an electronic gage.

Clause 25. The metrology and adjustment apparatus of any of clauses 13-24, wherein a positioning mechanism of the plurality of positioning mechanisms comprises a lockable adjustment handle.

Clause 26. The metrology and adjustment apparatus of any of clauses 13-25, wherein a positioning mechanism of the plurality of positioning mechanisms comprises a pneumatic motor or an electric motor.

Clause 27. The metrology and adjustment apparatus of any of clauses 13-26, wherein the first securing component or the second securing component comprises a holding element shaped and dimensioned to mate with an alignment feature of the medical device.

Clause 28. The metrology and adjustment apparatus of any of clauses 13-27, wherein the plurality of positioning mechanisms is configured to provide three degrees of freedom of angular orientation between the first securing component and the second securing component relative to each other.

Clause 29. The metrology and adjustment apparatus of any of clauses 13-28, further comprising a networking and control system configured to actuate the plurality of positioning mechanisms.

Clause 30. A method of adjusting a medical device, the method comprising: providing a metrology and adjustment apparatus having a first securing component, a second securing component, and a heater between the first securing component and the second securing component, wherein the first securing component and the second securing component are spaced apart in a Z-direction; securing a first region of the medical device with the first securing component; securing a second region of the medical device with the second securing component; positioning a heating zone of the medical device within the heater; heating the heating zone of the medical device; and adjusting a plurality of positioning mechanisms coupled to the first securing component and the second securing component to thermoform the medical device, wherein the plurality of positioning mechanisms are configured to provide: i) translation in an X-direction, a Y-direction, and the Z-direction for a relative position of the first securing component and the second securing component with respect to each other; and ii) at least two degrees of freedom of angular orientation between the first securing component and the second securing component relative to each other.

Clause 31. The method of clause 30, wherein the adjusting the plurality of positioning mechanisms is performed prior to the heating to provide a preloading force to the medical device.

Clause 32. The method of any of clauses 30-31, wherein the adjusting the plurality of positioning mechanisms comprises adjusting a distance between the first securing component and a center of rotation of the first securing component.

Clause 33. The method of any of clauses 30-32, wherein the adjusting comprises using a networking and control system to actuate the plurality of positioning mechanisms.

Clause 34. A method of adjusting a medical device, the method comprising: a) providing a metrology and adjustment apparatus having: a first securing component; a second securing component, wherein the first securing component and the second securing component are spaced apart in a Z-direction; a plurality of positioning mechanisms coupled to the first securing component and the second securing component, wherein the plurality of positioning mechanisms are configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other; and an arm coupled to the first securing component, wherein the arm is configured to allow adjustment of a distance between the first securing component and a center of rotation of the first securing component; b) securing a first region of the medical device with the first securing component; c) securing a second region of the medical device with the second securing component; and d) adjusting the medical device using the plurality of positioning mechanisms.

Clause 35. The method of clause 34, wherein the adjusting comprises using a networking and control system to actuate the plurality of positioning mechanisms.

Clause 36. The method of any of clauses 34-35, further comprising heating a heating zone of the medical device using a heater of the metrology and adjustment apparatus, the heater located between the first securing component and the second securing component.

Clause 37. A metrology and adjustment apparatus for a medical device, the apparatus comprising: a first securing component configured to hold a first region of the medical device; a second securing component configured to hold a second region of the medical device, the first securing component and the second securing component being spaced apart in a Z-direction; a plurality of positioning mechanisms coupled to at least one of the first securing component or the second securing component, the plurality of positioning mechanisms being configured to provide a combined total of at least six degrees of freedom between the first securing component and the second securing component, wherein the six degrees of freedom comprise translational movement in an X-direction, a Y-direction, and the Z-direction and rotational movement about an X-axis, a Y-axis, and a Z-axis; and a heater positioned between the first securing component and the second securing component.

Clause 38. The metrology and adjustment apparatus of clause 37, wherein the medical device is a prosthesis or an orthosis for a leg.

Clause 39. The metrology and adjustment apparatus of any of clauses 37-38, wherein the heater comprises a channel formed by lateral walls of a perimeter of a chamber of the heater.

Clause 40. The metrology and adjustment apparatus of any of clauses 37-39, wherein the heater comprises an electric heater integrated within a chamber of the heater.

Clause 41. The metrology and adjustment apparatus of any of clauses 37-39, wherein the heater comprises an electric heater that is external to a chamber of the heater and is used to heat a fluid to heat the medical device via convective heat transfer.

Clause 42. The metrology and adjustment apparatus of any of clauses 37-41, wherein a distance between the first securing component and a center of rotation of the first securing component is adjustable.

Clause 43. The metrology and adjustment apparatus of any of clauses 37-42, wherein the plurality of positioning mechanisms comprises a linear translation stage coupled to the first securing component or the second securing component, the linear translation stage configured to provide the translation in the X-direction or the Y-direction.

Clause 44. The metrology and adjustment apparatus of any of clauses 37-43, wherein the plurality of positioning mechanisms comprises a Z-translation stage coupled to the first securing component or the second securing component, the Z-translation stage configured to provide the translation in the Z-direction.

Clause 45. The metrology and adjustment apparatus of clause 44, wherein the Z-translation stage is mounted on a pair of rails.

Clause 46. The metrology and adjustment apparatus of any of clauses 37-45, wherein: the plurality of positioning mechanisms comprises an X-rotation mechanism, a Y-rotation mechanism, and a Z-rotation mechanism configured to achieve three degrees of freedom of rotation of the first region or the second region; the X-rotation mechanism is configured to rotate the first securing component or the second securing component about an X-axis that is oriented in the X-direction; the Y-rotation mechanism is configured to rotate the first securing component or the second securing component about a Y-axis that is oriented in the Y-direction; and the Z-rotation mechanism is configured to rotate the first securing component or the second securing component about a Z-axis that is perpendicular to a plane formed by the X-axis and the Y-axis.

Clause 47. The metrology and adjustment apparatus of clause 46, further comprising an arm that couples the X-rotation mechanism or the Y-rotation mechanism to the first securing component, wherein the arm is configured to allow adjustment of the distance between the first securing component and the center of rotation.

Clause 48. The metrology and adjustment apparatus of clause 47, wherein the arm comprises a plurality of holes along a length of the arm.

Clause 49. The metrology and adjustment apparatus of clause 47, wherein the center of rotation is an X-center of rotation of the X-rotation mechanism or a Y-center of rotation of the Y-rotation mechanism.

Clause 50. The metrology and adjustment apparatus of any of clauses 37-49, wherein the X-center of rotation or the Y-center of rotation is offset from a centroid of axes of rotation.

Clause 51. The metrology and adjustment apparatus of any of clauses 37-50, wherein the first securing component or the second securing component comprises a holding element shaped and dimensioned to mate with an alignment feature of the medical device.

In some cases, a single example may, for succinctness and/or to assist in understanding the scope of the disclosure, combine multiple features. It is to be understood that in such a case, these multiple features may be provided separately (in separate examples), or in any other suitable combination. Alternatively, where separate features are described in separate examples, these separate features may be combined into a single example unless otherwise stated or implied. This also applies to the claims which can be recombined in any combination. That is, a claim may be amended to include a feature defined in any other claim. Furthermore, a phrase referring to “at least one of” a list of items refers to any combination of those items, including single members. As an example, “at least one of: a, b, or c” is intended to cover: a, b, c, a-b, a-c, b-c, and a-b-c.

Reference has been made in detail to aspects of the disclosed invention, one or more examples of which have been illustrated in the accompanying figures. Each example has been provided by way of explanation of the present technology, not as a limitation of the present technology. In fact, while the specification has been described in detail with respect to specific aspects of the invention, it will be appreciated that those skilled in the art, upon attaining an understanding of the foregoing, may readily conceive of alterations to, variations of, and equivalents to these aspects. For instance, features illustrated or described as part of one aspect may be used with another aspect to yield a still further aspect. Thus, it is intended that the present subject matter covers all such modifications and variations within the scope of the appended claims and their equivalents. These and other modifications and variations to the present invention may be practiced by those of ordinary skill in the art, without departing from the scope of the present invention, which is more particularly set forth in the appended claims. Furthermore, those of ordinary skill in the art will appreciate that the foregoing description is by way of example only and is not intended to limit the invention.

What is claimed is:

1. A metrology and adjustment apparatus for a medical device, the apparatus comprising:
a first securing component configured to hold a first region of the medical device;
a second securing component configured to hold a second region of the medical device, the first securing component and the second securing component being spaced apart in a Z-direction;
a plurality of positioning mechanisms coupled to at least one of the first securing component or the second securing component, the plurality of positioning mechanisms being configured to provide a combined total of at least six degrees of freedom between the first securing component and the second securing component, wherein the six degrees of freedom comprise translational movement in an X-direction, a Y-direction, and the Z-direction and rotational movement about an X-axis, a Y-axis, and a Z-axis; and
a heater positioned between the first securing component and the second securing component.

2. The metrology and adjustment apparatus of claim 1, wherein the medical device is a prosthesis or an orthosis for a leg.

3. The metrology and adjustment apparatus of claim 1, wherein the heater comprises a channel formed by lateral walls of a perimeter of a chamber of the heater.

4. The metrology and adjustment apparatus of claim 1, wherein the heater comprises an electric heater integrated within a chamber of the heater.

5. The metrology and adjustment apparatus of claim 1, wherein the heater comprises an electric heater that is external to a chamber of the heater and is used to heat a fluid to heat the medical device via convective heat transfer.

6. The metrology and adjustment apparatus of claim 1, wherein a distance between the first securing component and a center of rotation of the first securing component is adjustable.

7. The metrology and adjustment apparatus of claim 1, wherein the plurality of positioning mechanisms comprises a linear translation stage coupled to the first securing component or the second securing component, the linear translation stage configured to provide the translational movement in the X-direction or the Y-direction.

8. The metrology and adjustment apparatus of claim 1, wherein the plurality of positioning mechanisms comprises a Z-translation stage coupled to the first securing component or the second securing component, the Z-translation stage configured to provide the translational movement in the Z-direction.

9. The metrology and adjustment apparatus of claim 8, wherein the Z-translation stage is mounted on a pair of rails.

10. The metrology and adjustment apparatus of claim 1, wherein:
the plurality of positioning mechanisms comprises an X-rotation mechanism, a Y-rotation mechanism, and a Z-rotation mechanism configured to achieve three degrees of freedom of rotation of the first region or the second region;
the X-rotation mechanism is configured to rotate the first securing component or the second securing component about the X-axis that is oriented in the X-direction;
the Y-rotation mechanism is configured to rotate the first securing component or the second securing component about the Y-axis that is oriented in the Y-direction; and
the Z-rotation mechanism is configured to rotate the first securing component or the second securing component about the Z-axis that is perpendicular to a plane formed by the X-axis and the Y-axis.

11. The metrology and adjustment apparatus of claim 10, further comprising an arm that couples the X-rotation mechanism or the Y-rotation mechanism to the first securing component, wherein the arm is configured to allow adjustment of a distance between the first securing component and a center of rotation of the first securing component.

12. The metrology and adjustment apparatus of claim 11, wherein the arm comprises a plurality of holes along a length of the arm.

13. The metrology and adjustment apparatus of claim 11, wherein the center of rotation is an X-center of rotation of the X-rotation mechanism or a Y-center of rotation of the Y-rotation mechanism.

14. The metrology and adjustment apparatus of claim 13, wherein the X-center of rotation or the Y-center of rotation is offset from a centroid of axes of rotation.

15. The metrology and adjustment apparatus of claim 13, wherein the first securing component or the second securing component comprises a holding element shaped and dimensioned to mate with an alignment feature of the medical device.

16. A metrology and adjustment apparatus for a medical device, the apparatus comprising:
a first securing component configured to hold a first region of the medical device;
a second securing component configured to hold a second region of the medical device, the first securing component and the second securing component being spaced apart in a Z-direction;
a plurality of positioning mechanisms configured to provide i) translation in an X-direction, a Y-direction, and the Z-direction for a relative position of the first securing component and the second securing component with respect to each other; and ii) at least two degrees of freedom of angular orientation between the first securing component and the second securing component relative to each other; and
a heater positioned between the first securing component and the second securing component;
wherein a distance between the first securing component and a center of rotation of the first securing component is adjustable.

17. The metrology and adjustment apparatus of claim 16, wherein the plurality of positioning mechanisms comprises a linear translation stage coupled to the first securing component or the second securing component, the linear translation stage configured to provide the translation in the X-direction or the Y-direction.

18. The metrology and adjustment apparatus of claim 16, wherein the plurality of positioning mechanisms comprises a Z-translation stage coupled to the first securing component or the second securing component, the Z-translation stage configured to provide the translation in the Z-direction.

19. The metrology and adjustment apparatus of claim 16, wherein:
the plurality of positioning mechanisms comprises an X-rotation mechanism, a Y-rotation mechanism, and a Z-rotation mechanism configured to achieve three degrees of freedom of rotation of the first region or the second region;
the X-rotation mechanism is configured to rotate the first securing component or the second securing component about an X-axis that is oriented in the X-direction;
the Y-rotation mechanism is configured to rotate the first securing component or the second securing component about a Y-axis that is oriented in the Y-direction; and
the Z-rotation mechanism is configured to rotate the first securing component or the second securing component about a Z-axis that is perpendicular to a plane formed by the X-axis and the Y-axis.

20. The metrology and adjustment apparatus of claim 19, further comprising an arm that couples the X-rotation mechanism or the Y-rotation mechanism to the first securing component, wherein the arm is configured to allow adjustment of the distance between the first securing component and the center of rotation.
